(19) United States
(12) Patent Application Publication
(10) Pub. No.: US 2024/0218360 A1
(43) Pub. Date: Jul. 4, 2024

(54) SMALL ACTIVATING RNA INCREASING SHANK EXPRESSION AND METHOD OF TREATING INTELLECTUAL DISABILITIES AND ASSOCIATED COMORBIDITIES ASSOCIATED WITH SHANK HAPLOINSUFFICIENCY

(71) Applicant: ITAYANDBIOND LTD, Tel Aviv-Yaffo (IL)

(72) Inventors: Shmulik BEZALEL, Kefar Vradim (IL); Jonatan DARR, Rehovot (IL); Nisim PERETS, Tel Aviv (IL)

(21) Appl. No.: 18/285,396

(22) PCT Filed: Apr. 3, 2022
(86) PCT No.: PCT/IL2022/050350

§ 371 Date: Oct. 3, 2023

Related U.S. Application Data
(60) Provisional application No. 63/170,627, filed on Apr. 5, 2021.

(51) Int. Cl. C12N 15/113

(52) U.S. Cl. CPC C12N 15/113; C12N 2310/11; C12N 2310/531

(57) ABSTRACT

The present invention provides saRNA including one strand having at least 75% homology or complementarity with any continuous fragment of 16 to 35 nucleotides in length of a promoter sequence of a human SHANK protein, wherein the saRNA activates or upregulates the expression of the SHANK protein by targeting the human SHANK promoter.

### FIELD OF THE INVENTION

The invention is directed to small activating RNAs increasing SHANK expression and method of treating intellectual disabilities associated with SHANK haploinsufficiency.

### BACKGROUND OF THE INVENTION

The prevalence of Autism Spectrum Disorders (ASD) is high and growing rapidly. According to a 2018 report from the Centre for Disease Control and Prevention (CDC), the incidence of ASD in children in the United States more than doubled from 1 in 125 in 2008 to 1 in 59 in 2018. ASD includes a range of neurodevelopmental disorders that affect social and communication skills. Raising children with ASD places huge demands on parents and school systems, and adults with ASD often have difficulty developing social relationships, maintaining jobs, and performing daily tasks. In addition, many ASD patients, in particular patients suffering from Phelan-McDermid syndrome, likewise endure various comorbidities, such as, but not limited to gastrointestinal disorders including chronic bowel inflammation disorders and other relevant comorvbidities.

The underlying basis of ASD is poorly understood, making ASD difficult both to diagnose and to treat. Although certain risk factors, such as high parental age and gestational diabetes, are associated with ASD, specific causes have not been identified. For example, autism displays a strong heritability component, but most cases cannot be linked to individual mutations. Thus, ASD is thought to result from multiple mutations that have low penetrance. In addition, many mutations that are associated with autism are not inherited from a parental genome but appear to have occurred during embryonic development. Therefore, ASD cannot be reliably predicted at an early stage from genetic data alone. Moreover, because the molecular mechanisms of ASD are not known, drugs to treat them are lacking. Existing pharmacological approaches are limited to the use of psychoactive or anticonvulsant medications to treat symptoms, such as irritability, self-injury, aggression, and tantrums, associated with ASD. However, such drugs do not remedy the social and communication impairments at the core of ASD.

Consequently, the tools to diagnose and treat ASD remain woefully inadequate even as increasing numbers of people are affected by these disorders.

The biggest evidence for a genetic contribution to ASD comes from twin studies, which show 31% concordance rates for dizygotic twins and 88% for monozygotic twins. ASDs are clinically heterogeneous, with symptom-severity ranging from mild social deficits with normal cognitive abilities to severe mental impairment and absence of language skills. Evidence points towards the involvement of a number of distinct genetic variations (including chromosomal rearrangements, copy number variations and coding sequence variants) that have been associated with many different genes.

### SUMMARY OF THE INVENTION

The present invention is directed to small activating RNAs which increase the expression of SHANK proteins as well as to methods of treating intellectual disabilities and/or associated comorbidities, in particular autism, associated with SHANK haploinsufficiency.

Several large-scale genomic studies have supported an association between cases of syndromic and idiopathic autism spectrum disorder, as well as between other neuropsychiatric and neurodevelopmental disorders (schizophrenia and intellectual disability) and mutations in multiple ankyrin repeat domains proteins 1-3 (SHANK1, SHANK2 and SHANK3 respectively), which encode a family of postsynaptic scaffolding proteins that are present at glutamatergic synapses in the CNS.

However, despite great research progress in establishing links between mutations in SHANK genes and ASD, the physiological role of SHANK proteins is poorly understood.

The herein disclosed invention advantageously utilizes RNA activation (RNAa) utilizing short dsRNAs termed small activating RNAs (saRNAs) targeting promoter-derived sequences of SHANK to induce/enhance its expression.

Traditionally, gain-of-function studies often require the use of an exogenous DNA construct for ectopic expression. Such systems typically do not resemble natural genes, in that they are often cloned from cDNA libraries or PCR amplicons that lack regulatory elements such as introns and untranslated regions (UTRs), which may be involved in multiple processes such as alternative splicing, post-transcriptional modification and transcript stability which in turn can influence gene function. Moreover, in the clinic, gene overexpression systems are problematic since they require viral-based systems to drive the delivery of the exogenous genes that may have detrimental effects on host genome integrity as well as pose undesired immunological consequences.

Advantageously, the herein disclosed saRNAs provide an alternative approach, which enables enhancing SHANK gene expression in a safe and controlled manner. Furthermore, the saRNAs enable selective upregulation of SHANK gene expression, without introducing exogenous DNA, thereby avoiding detrimental position effects, unanticipated dysregulation of other genes as well as a harmful immunological response.

According to some embodiments, there is provided a saRNA, wherein one strand of the saRNA has at least 75% homology or complementarity with any continuous fragment of 16 to 35 nucleotides in length of a promoter sequence of a human SHANK protein, and wherein the saRNA activates or upregulates the expression of the SHANK protein by targeting the human SHANK promoter.

According to some embodiments, the saRNA comprises a sense nucleic acid strand and an antisense nucleic acid strand, the sense nucleic acid strand and the antisense nucleic acid strand contain complementary regions capable of forming a double-stranded nucleic acid structure, and the sense nucleic acid strand or the antisense nucleic acid strand has more than 75%, more than 80%, more than 90%, more than 95%, more than 99%, or 100% homology with any continuous fragment of 16 to 35 nucleotides in length in a sequence of the human SHANK promoter.

According to some embodiments, the sense nucleic acid strand and the antisense nucleic acid strand are on two different nucleic acid strands.

According to some embodiments, the sense nucleic acid strand and the antisense nucleic acid strand are on the same nucleic acid strand, forming a hairpin single-stranded nucleic acid molecule, wherein the complementary regions of the sense nucleic acid strand and the antisense nucleic acid strand form a double-stranded nucleic acid structure.

According to some embodiments, at least one strand of the saRNA has a 3′ overhang of 0 to 6 nucleotides in length. According to some embodiments, both strands of the saRNA have a 3′ overhang of 2 to 3 nucleotides in length.

According to some embodiments, the sense nucleic acid strand or the antisense nucleic acid strand is 16 to 35 nucleotides in length.

According to some embodiments, the sense strand has a nucleotide sequence having at least 75% sequence homology to any one of the nucleotide sequences set forth in any of SEQ ID NO: 1-3. Each possibility is a different embodiment.

According to some embodiments, the antisense strand comprises a nucleotide sequence having at least 90% sequence homology to any one of the nucleotide sequences set forth in SEQ ID NO: 4-46. Each possibility is a different embodiment.

According to some embodiments, the promotor targeted by the saRNA has a nucleotide sequence with at least 75% sequence homology to the nucleotide sequence set forth in SEQ ID NO: 1 and the antisense strand comprises a nucleotide sequence having at least 90% sequence homology to any one of the nucleotide sequences set forth in SEQ ID NO: 4-24, or in SEQ ID NO: 81-83. Each possibility is a different embodiment.

According to some embodiments, the promotor targeted by the saRNA has a nucleotide sequence with at least 75% sequence homology to the nucleotide sequence set forth in SEQ ID NO: 1 and wherein the antisense strand comprises a nucleotide sequence having at least 90% sequence homology to any one of the nucleotide sequences set forth in SEQ ID NO: 6, 7, 8, 10, 81, 82, and 83. Each possibility is a different embodiment.

According to some embodiments, the promotor targeted by the saRNA has a nucleotide sequence with at least 75% sequence homology to the nucleotide sequence set forth in SEQ ID NO: 2 and the antisense strand comprises a nucleotide sequence having at least 90% sequence homology to any one of the nucleotide sequences set forth in SEQ ID NO: 25-35. Each possibility is a different embodiment.

According to some embodiments, the promotor targeted by the saRNA has a nucleotide sequence with at least 75% sequence homology to the nucleotide sequence set forth in SEQ ID NO: 3 and wherein the antisense strand comprises a nucleotide sequence having at least 90% sequence homology to any one of the nucleotide sequences set forth in SEQ ID NO: 36-46. Each possibility is a different embodiment.

According to some embodiments, the SHANK protein is SHANK 3.

According to some embodiments, the antisense strand further comprises a 3′ having a length of 2-15 nucleotides.

According to some embodiments, the antisense strand comprises a nucleotide analogue.

According to some embodiments, the saRNA may further include 5-10 additional 3′ and/or 5′ base pairs in addition to the core 18-21 bases. In some embodiments, the saRNA may further include 2-5 additional 3′ and/or 5′ base pairs in addition to the core 18-21 bases.

According to some embodiments, certain bases of the saRNA may optionally be substituted.

According to some embodiments, the saRNA further comprises a nuclear localization sequence.

According to some embodiment, the nuclear localization signal may enhance the efficacy of the saRNA, as exemplified by the saRNA having an antisense strand nucleotide sequence as set forth in SEQ ID NO: 82 and SEQ ID NO: 83 that comprise nuclear localization sequences or part thereof.

According to some embodiments, there is provided a composition comprising one or more of the saRNA molecules described herein, and a suitable transport vehicle and/or carrier.

According to some embodiments, the carrier is an aqueous solution.

According to some embodiments, the composition is suitable for administration through aerosol.

According to some embodiments, the transport vehicle is a liposome, a conjugated peptide or protein, a delivery molecule, an exosome, a nanoparticle (for example, a polymeric and/or lipid-based nanoparticle), dendrimers, micelles, nanoemulsions and nanosuspensions, a microspheres or cells.

According to some embodiments, the composition is formulated for oral and/or nasal administration. According to some embodiments, the composition is formulated for administration via inhalation. According to some embodiments, the composition is formulated for intranasal and/or intrabuccal administration.

According to some embodiments, the composition comprises at least two different saRNA molecules.

According to some embodiments, there is provided a method for treating/ameliorating/preventing an intellectual disability, the method comprising administering to a subject in need thereof the saRNA and/or compositions described herein, thereby treating/ameliorating/preventing the intellectual disability.

According to some embodiments, the intellectual disability is Phelan-McDermid syndrome (PMS) and the method treats and/or ameliorates the symptoms of Phelan-McDermid syndrome.

According to some embodiments, the intellectual disability is idiopathic autism spectrum disorder (ASD) and the method treats and/or ameliorates the symptoms of the ASD.

According to some embodiments, the intellectual disability is schizophrenia, and the method treats and/or ameliorates the symptoms of the schizophrenia.

According to some embodiments, the subject is a normal subject predisposed to suffer from the intellectual disability.

According to some embodiments, the method further includes treating a digestive disorder associated with the intellectual disability.

According to some embodiments, there is provided use of the herein disclosed saRNA and/or compositions, for treating, ameliorating and/or preventing an intellectual disability of a subject.

According to some embodiments, there is provided a method for treating/ameliorating/preventing an intellectual disability associated comorbidity, the method comprising administering to a subject in need thereof the saRNA and/or the composition described herein, thereby treating/ameliorating/preventing the intellectual disability comorbidity.

According to some embodiments, the intellectual disability is Phelan-McDermid syndrome (PMS).

According to some embodiments, the intellectual disability associated morbidity is chronic bowel inflammation.

According to some embodiments, double-stranded RNA molecules corresponding to SEQ ID NO: 6, 7, 8, 10, 81, 82, and 83 upregulate the activity of promoter 1.

According to some embodiments, double-stranded RNA molecules corresponding to SEQ ID NO: 6, SEQ ID NO: 8, SEQ ID NO: 10 SEQ ID NO: 81, SEQ ID NO: 82, and SEQ ID NO: 83 increased SHANK3 mRNA levels by 1.2-1.4-fold, relative to untransfected cells.

According to some embodiments, double-stranded RNA molecules corresponding to SEQ ID NO: 7 exhibited a much stronger upregulation effect, advantageously, reaching up to 2.1- and 1.8-fold change, relative to untransfected cells.

According to some embodiments, double-stranded RNA molecule corresponding to SEQ ID NO: 7 exhibited upregulation of SHANK3 mRNA levels well above the 1.4-fold change that was expected from treatment with the positive control lithium bicarbonate that exerts a completely different, unspecific, more global effect on gene expression.

According to some embodiments, double-stranded RNA molecule corresponding to SEQ ID NO: 7 exhibited upregulation of SHANK3 mRNA levels by more than two-fold as compared to untreated control cells and by 1.5 fold as compared to the positive control (lithium).

According to some embodiments, activity of double stranded RNA molecules capable of enhancing SHANK3 levels (i.e., saRNA) is performed in human or optionally as a preliminary assay in mouse cell lines such as but not limited to GDM1, KG1, and/or terminally differentiated neurons derived from iPSCs from neurotypical, idiopathic ASD, and Shank3 ASD patients.

According to some embodiments, the saRNA is delivered in-vivo to the brain of an animal. According to some embodiments, the saRNA is conjugated to a delivery vehicle that transports the double-stranded RNA across the blood-brain barrier.

According to some embodiments, the delivery vehicle that transports the double-stranded RNA across the blood-brain barrier is a delivery vehicle capable of carrying oligonucleotides across the blood-brain barrier.

According to some embodiments, the delivery vehicle capable of carrying oligonucleotides across the blood-brain barrier includes the following non-limiting examples, a nanocarrier consisting of gold, silica or iron, a cationic polymer such as polyethyleneimine (PEI), poly-(lactic coglycolic acid) PLGA, chitosan or collagen, a protein nanoparticles (human serum albumin) coated with apolipoprotein-A, or a cationic liposome or any combination thereof. Each possibility is a separate embodiment.

Certain embodiments of the present disclosure may include some, all, or none of the above advantages. One or more technical advantages may be readily apparent to those skilled in the art from the FIGURES, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

FIG. 1A. presents FACS results of transfection efficiency in KG-1 cells. results presented are of saRNA blocked with BLOCK-iT fluorescent oligo (FITC)—reagent for optimization of transfection of small oligonucleotides, relative to untreated cells (UN). Analyses was performed using FF-100 program of the Nucleofector 4D electroporation system by Lonza.

FIG. 1B. presents qRT-PCR results of SHANK3 expression in KG-1 cells. The change in Shank3 mRNA level was measured 72 h after cells were transfected and treated either with 1 uM and/or 2 uM of different double-stranded RNA molecules having the nucleotide sequences set forth in: SEQ ID NO: 6, 7, 8, 10, 81, 82 and 83 targeting promoter 1 of SHANK3, or with 1 uM siRNA or 0.5 mM lithium bicarbonate, both serving as positive controls for down- and up-regulation of SHANK3 expression, respectively, and compared to untreated cells (UN). Results are normalized to GAPDH.

FIG. 1C. presents FACS results of KG-1 cells viability as assayed by live/dead staining. viability was estimated 72 h after cells were transfected and treated either with 1 uM and/or 2 uM of 8 different double-stranded RNA molecules having the nucleotide sequences set forth in: SEQ ID NO: 6, 7, 8, 10, 81, 82, and 83, and potentially targeting promoter 1 of SHANK3, or with 1 uM siRNA or 0.5 mM lithium bicarbonate, both serving as positive controls for down- and up-regulation of Shank3 expression, respectively, and compared to untreated cells (UN).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

As used herein, a “nucleotide” comprises a nitrogenous base, a sugar molecule, and a phosphate group. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, locked nucleic acids, arabinose, and hexose). According to some embodiments, the sugar and/or phosphate groups may be modified to include a peptide bond, so as to obtain a Peptide Nucleotide Acid (PNA).

As used herein, the term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript” are synonymous as used herein.

As used herein, the term “small activating RNA” (“saRNA”), also referred to in the art as refers to an RNA (or RNA analog) comprising between about 15-25 nucleotides (or nucleotide analogs) that is capable of targeting a promoter of a gene and as a result induce and/or enhance gene expression from the promoter. In certain embodiments, the 3′ end of the saRNA molecules may include additional nucleotides that create an overhang, such as, but not limited to “TT”.

As used herein, the term “short hairpin RNA” (“shRNA”) refers to an saRNA precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAa, and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into saRNAs by the conserved cellular Argonaute-mediated machinery. saRNAs are capable of activating expression of a target gene through the complementarity of the “guide strand” portion of the saRNA to the promoter of the target gene. In certain embodiments of the invention the 5′ end of an shRNA has a phosphate group while in other embodiments it does not. In certain embodiments of the invention the 3′ end of an shRNA has a hydroxyl group.

As used herein, the term “RNAa-inducing vector” includes a vector whose presence within a cell results in transcription of one or more RNAs that self-hybridize or hybridize to each other to form an RNAa molecule. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in the production of one or more RNAs that self-hybridize or hybridize to each other to form an RNAa molecule. In general, the vector comprises a nucleic acid operably linked to expression signal(s) so that one or more RNA molecules that hybridize or self-hybridize to form an RNAa molecule is transcribed when the vector is present within a cell. Use of the term “induce” indicates that presence of the vector within a cell results in production of an RNAa agent within the cell, leading to an RNAa-mediated enhancement in the expression of a gene, the promoter of which the RNAa molecule is targeted.

An RNAa-inducing entity is considered to be targeted to a target promoter for the purposes described herein if (1) the agent comprises a strand that is substantially complementary to the promoter sequence over 15-29 nucleotides, e.g., 15, more preferably at least about 17, yet more preferably at least about 18 or 19 to about 21-23 or 24-29 nucleotides. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% sequence complementarity/homology with the target promoter over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAa agent hybridizes to the promoter sequence under stringent conditions for hybridization of small (<50 nucleotide) RNA molecules in vitro and/or under conditions typically found within the cytoplasm or nucleus of mammalian cells.

As used herein, the term “complementary” refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. According to some embodiments, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may have may contain 0-5 mismatches within the window; if the window is 19 nucleotides long, substantially complementary nucleic acids may contain 0-6 mismatches within the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0).

According to some embodiments, the RNAa molecules disclosed herein may be purified. Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

As used herein, the term “nuclear localization sequence” refers to short consensus sequences encoded capable of bringing about transport of nucleic acid molecules including RNAs from the cytoplasm and into the nucleus. According to some embodiments, and without being bound by any theory, the nuclear localization sequence binds to a nuclear RNA-binding protein, such as, but not limited to, RNA binding protein heterogeneous nuclear ribonucleoprotein K (HNRNPK) associated with accumulation of RNAs into the nucleus.

According to some embodiments, the nuclear localization signal may include three stretches of six pyrimidines within a 42 nt sequence context, containing the sequence RCCTCCC (where R stands for A or G) at least twice. According to some embodiments, the nuclear localization signal may have the sequence AGUGUU. According to some embodiments, the AGUGUU nuclear localization signal may be positioned at the 3′ of the saRNA.

As used herein, the term “gene” refers to all nucleotide sequences required to encode a polypeptide chain or to transcribe a functional RNA. “Gene” can be an endogenous or fully or partially recombinant gene for a host cell (for example, because an exogenous oligonucleotide and a coding sequence for coding a promoter are introduced into a host cell, or a heterogeneous promoter adjacent to an endogenous coding sequence is introduced into a host cell). For example, the term “gene” includes a nucleic acid sequence composed of exons and introns. Protein-coding sequences are, for example, sequences contained within exons in an open reading frame between an initiation codon and a termination codon, and as used herein, “gene” can comprise a gene regulatory sequence, such as a promoter, an enhancer, and all other sequences known in the art for controlling the transcription, expression or activity of another gene, no matter whether the gene contains a coding sequence or a non-coding sequence. In one case, for example, “gene” can be used to describe a functional nucleic acid containing a regulatory sequence such as a promoter or an enhancer. The expression of a recombinant gene can be controlled by one or more types of heterogenous regulatory sequences.

The term “target gene” as used herein can refer to nucleic acid sequences, transgenes, viral or bacterial sequences, chromosomes or extrachromosomal genes that are naturally present in organisms, and/or can be transiently or stably transfected or incorporated into cells and/or chromatins thereof. The target gene can be a protein-coding gene or a non-protein-coding gene (such as microRNA gene and long non-coding RNA gene). The target gene generally contains a promoter sequence, and the positive regulation for the target gene can be achieved by designing a saRNA having sequence homology with the promoter sequence, characterized as the upregulation of expression of the target gene. “Sequence of a target gene promoter” refers to a non-coding sequence of the target gene, and the reference of the sequence of a target gene promoter in the phrase “complementary with the sequence of a target gene promoter” of the present invention means a coding strand of the sequence, also known as a non-template strand, i.e. a nucleic acid sequence having the same sequence as the coding sequence of the gene. “Target sequence” refers to a sequence fragment in the target gene promoter sequence, which is homologous or complementary with a sense oligonucleotide strand or an antisense oligonucleotide strand of a saRNA.

As used herein, the terms “sense strand”, “sense oligonucleotide strand” and “passenger strand” may be used interchangeably and refer to a having homology with the coding strand of the promoter sequence of the target gene in the saRNA duplex.

As used herein, the terms “antisense strand”, “antisense oligonucleotide strand” and “guide strand” can be used interchangeably and refer to a ribonucleic acid strand complementary with the sense oligonucleotide strand in the saRNA duplex and with the target promoter sequence.

According to some embodiments, the guide strand may include a nucleotide sequence having at least 75%, at least 80%, at least 85%, at least 90%, at least 98% or 100% sequence homology to any one of the nucleotide sequences set forth in Table 1-5 below.

TABLE 1
saRNA guide strand sequences targeting promoter 1 (SEQ ID NO: 1) of SHANK3

| SEQ ID | | SEQ ID | |
|---|---|---|---|
| NO: | Guide strand sequence | NO: | Guide strand sequence |
| 4 | UUUCUGAUGAGCCCUCUCC | 15 | AACUGUGGUCCCAGCAUCA |
| 5 | UAAACACCCUGACCCACAC | 16 | UAACCUUGAACUGCUCCGG |
| 6 | UAGUUUGGGGAGUUUGCUC | 17 | GAACGUUCACAGAAGAGCC |
| 7 | CAAUUAAUUUCCGGAGGUG | 18 | AACGGAUUUUGCCUGAGCC |
| 8 | UAAAGAAACGAGCUCAGGC | 19 | GAUCACCCACAAAACGGAU |
| 9 | GUCGAAAUAGUUCCGCCUC | 20 | AAACAGCCUCUAACCCCCA |
| 10 | UAACAAUACCCACAAGCAC | 21 | CAAUUAAUUUCCGGAAGUG |
| 11 | UUGAUGGUCCCCAGACUCA | 22 | UAAAGAAACGAGCUCAGUG |
| 12 | GAGUCCAAGCCUGAGUCAC | 23 | AACGGAUUUUGCCUGAGUG |
| 13 | CACAUCUUCACCAACCAGC | 24 | GAACGUUCACAGAAGAGUG |
| 14 | UAACCCUAACCUUCCUUGC | 81 | UUGUCGUGGCAACGCGGGA |
| 82 | AGUAAAGAAACGAGCUCAGUG | 83 | GAGAACGUUCACAGAAGAGUG |

TABLE 2
saRNA guide strand sequences targeting promoter
2 (SEQ ID NO: 2) of SHANK3 strand

| SEQ ID NO: | Guide strand sequence |
|---|---|
| 25 | UUGAGGUUCAGGAAUCUGA |
| 26 | UAGAGCAAAGACAGUGGAA |
| 27 | UAUGAGCCCACUGUGUGCA |
| 28 | UAGAUGCUGUGCUCAUGCA |
| 29 | UAAGUAGCUGCUGAACCAG |
| 30 | UAGGGAAUUCUGACCUCCG |
| 31 | GAAUCUGCAUUACCCACAC |
| 32 | GUGUCCAGAUGGAAGUAGG |
| 33 | GAAGCUGAAGACAUCCCUG |
| 34 | UUAGGAAGGCGCUUCACCA |
| 35 | CAAAGGUCUAAGGUCAGGG |

TABLE 3
saRNA guide strand sequences targeting promoter
3 (SEQ ID NO: 3) of SHANK3 strand

| SEQ ID NO: | Guide strand sequence |
|---|---|
| 36 | UUAUCCACUCACACCCUUU |
| 37 | AAGCACACUGAUGAGUUGC |
| 38 | UUUCCCAGGCCUCUUCCUU |
| 39 | UUUGAAUCCUGCACCGUCU |
| 40 | UUCCAUGUUUCCAUGGCCU |
| 41 | UAAGCUGUCUUUGCUCCCA |
| 42 | UUGUCUCCCACCUGUGAGA |
| 43 | UUGUGCCUGUCUGGGUAGU |
| 44 | UUCAGUGGAGUUGUGAUGG |
| 45 | CAUGAUACUUCACCAGGCA |
| 46 | CAAGUGCAGGUGAACCACA |

TABLE 4
saRNA guide strand sequences targeting promoter
4 (SEQ ID NO: 47) of SHANK3

| SEQ ID NO: | Guide strand sequence |
|---|---|
| 49 | UUUUAACCAGCUACCUGCA |
| 50 | UUCCUUCCUUUCACCAGGG |
| 51 | UUACUCUGGCCUGUGAUUC |
| 52 | UUAGUGAAUCUCACAGUGC |
| 53 | UAUUUCCAGCUCCCCUUUC |
| 54 | UUAGGGUACCCUAGGGCUU |
| 55 | UUCAUGACUCCAUAUAGGG |
| 56 | UUCAGGCUUACUAUCCCAA |
| 57 | UAGACAUGUCCUUGACCUG |
| 58 | UUGCCCUACUCGGUAGUGU |
| 59 | GACAAGCAGGAAAAUUGGU |
| 60 | UACCCUGGUCCUAUACACG |
| 61 | UAUUCCUUCCUUUCACCAGUG |
| 62 | GUUUACUCUGGCCUGUGAGUG |
| 63 | GUUUCAUGACUCCAUAUAGUG |
| 64 | UGUAGACAUGUCCUUGAAGUG |
| 65 | GAAUUGCCCUACUCGGUAGUG |
| 66 | |

TABLE 5
saRNA guide strand sequences targeting promoter
5 (SEQ ID NO: 48) of SHANK3

| SEQ ID NO: | Guide strand sequence |
|---|---|
| 67 | UUUUAACAGGCAACCUCUG |
| 68 | UUCCAACCAAUUUGCCACC |
| 69 | UACUGUUACUCUACAGGGC |
| 70 | UUAGAAAACCCAAAGGGCC |
| 71 | UAUUCUGUGCCAACUGCAA |
| 72 | UUUGCCCCUUGUAGGUUUC |
| 73 | UAAGGAGGUCGAUACUGCA |
| 74 | UACUGGUAGAGAAACCAGC |
| 75 | UAUACCUGGCUCUGGAGAA |
| 76 | CAAUCUAUGGUGACCACUU |
| 77 | UUUUAACAGGCAACCAGUG |
| 78 | UAUACCUGGCUCUGGAGUG |
| 79 | CAAUCUAUGGUGACCAGUG |
| 80 | |

The term “coding strand” as used herein refers to a DNA strand in the target gene which cannot be used for transcription, and the nucleotide sequence of this strand is the same as that of RNA produced from transcription (in the RNA, T in DNA is replaced by U). The coding strand of the double-stranded DNA sequence of the target gene promoter described in the present invention refers to a promoter sequence on the same DNA strand as the DNA coding strand of the target gene.

The term “template strand” as used herein refers to the other strand complementary with the coding strand in the double-stranded DNA of the target gene, i.e. the strand that, as a template, can be transcribed into RNA, and this strand is complementary with the transcribed RNA (A to U, G to C). In the process of transcription, RNA polymerase is bound with the template strand, moves along the 3′ to 5′ direction of the template strand, and catalyzes the synthesis of the RNA along the 5′ to 3′ direction. The template strand of the double-stranded DNA sequence of the target gene promoter described in the present invention refers to a promoter sequence on the same DNA strand as the DNA template strand of the target gene.

The term “promoter” as used herein refers to a nucleic acid sequence, which does not encode a protein, which plays a regulatory role for the transcription of a protein-coding or RNA-coding nucleic acid sequence by associating with them spatially. Generally, a eukaryotic promoter contains 100 to 5,000 base pairs, although this length range is not intended to limit the term “promoter” as used herein. Although the promoter sequence is generally located at the 5′ terminus of a protein-coding or RNA-coding sequence, in some cases, the promoter sequence also exists in exon and intron sequences.

The promoter sequences target by the herein disclosed saRNAs are as set forth.

Promoter 1-
SEQ ID NO: 1
GATGGGAGAGAAGAGGGGGTAGACCTGGGCCCAGGGTGGCCCTGCCTGGCC
GGTGGGGTCTGGTGGGCCAGAGGGAGGGTCCTCAGGAAGCTTGGGGAGGCCGTGGG
GAACCTGGGGATTATTCTCAACAAAGTAAGGGGCAAAGACATCCTGTGTCCCTTGGC
TTCCTCTCCAGGAACCCCCCCGGAGCCCAGAGGACGTCATGTGTGTGAGTGCTTGTG
GGTATTGTTATTTTTGCTGTTTGCCGGGGTGGCGGTGGAGCTGGGGAGTGACTCAGG
CTTGGACTCTTTTCACGATGTGAGCATCATGCGTGCCAGGCACTGTGCAGAACCCAG
GCTCAGGGCGGCACTGGGCAGGGGCTACGGTGTTGCCCCGGGCAGTGGGTCTTTGA
CCCAGCTGAGTCTGGGGACCATCAATCTGTGGGCTGCTGGTTGGTGAAGATGTGAGA
TTTTCTCTCAAAATGCTCAGTGGCCACAGCCCCAGTGCTGGCCTGGGGTGGCTGGAT
TCACCTGGGCTGGTGGGTTGGCAGGAGGCGGCTGGTGGGGCCCAGAGGTCCAGTCT
AAGGGTCGGAGGAGTGCCGGCCAGGGTCTCTGGGTCCACCCAGCCGAGGGCAGGCT
TGCAGGGAGACACCAAGGGAGAGGGAGGAGGCCCAGGGCTGTGGAAGACCTGGCT
GCGGAGAGTCCATCACTGGTGGACTGGGAGGCCCCATCCACCACCTTCTGCTGGCCA
CAGTGGGATGAAGGGCCGGCACCTGGGAGCAAAGGCAGGTGGAGGTTGCGGTGGG
AGGGACGGCAAGGAAGGTTAGGGTTAGGGCTGAAGGAGGGGTTGGGAGGGAGTTG
GGGGCCTGGGTGACAGCGGGGGGAGAGACATGGGCAGGAAGACGGTGGAGACACG
AGGTGGGCCGGGAAGCACTGGTTTCCTCTCTCCAGGGCTGGGCCACAGGAAATCCA
GCCCTCGTGCCTTCCAAGGTGCTTCCTGCCGATACGGGAGCCTCCAGGGCCCCTTCC
TGCTCCCTGCTCCCGGGACCCCCACCCCTCCCATCCTGCCCCCTCCCCTGGGAGACC
CTCAGCTGCACCACTCAGGCCAGGCCAGTGGCCTTGGGAGGGGCCTGTGATGCTGG
GACCACAGTTCCTGGGCAGGGAGCAACCGTCTAGGCGTGGGGAGAACGCAGGACGT
GACCCACACACCGCACTGGAGGCTCCGCTCTGCCCGGTCAGTGGCGTCCTGGGGAG
GCGGGAGGAGGAAGCCAGACTGGTCCCAGGGAAGTGAGGCCCTGCTCTGGGGTTGA
CCAGAGGCTGTTTTGAGGGGAGAGGGCTCATCAGAAACACTCTGTCAGGGGTGCTC
TGGGGTGAGCAGGTGCTGGTCGAGGTGGATCTGCTGGGGAGTGGGGGCTGTCCTTG
GAGGGAGTGCCAAGCTCTGTTCTGTGTGGGTCAGGGTGTTTAGGATGGTCCAGGTTG
TCACGCGGGGGAGGGTAGGGAGGTTTTTCTGCGTGTATGGGGGCCGGAGCAGTTCA
AGGTTATTCCTGGGAGGCTCAGGCAAAATCCGTTTTGTGGGTGATCTGGAGCTGCTC
TTGGGGAGGCTGGGGGTTAGAGGCTGTTTTCCTCCTTAGGCAGAGGGGTTCCCCAGG
CTGGGCTCTTCTGTGAACGTTCTCAAGCTCTGCCCTCAGGGGGCCAGCGGCTCCTCT
AGGAGGATCTGGGGCTGCTTGGGGGCGGAGGAGGGGGGTGGGGAACGCCAGGCAA
AGCGGGTTGGGAGAGTTCTGCAGGAGGCGGAACTATTTCGACTTAGGGGCTGCTCTT
GGGGGCACAGGGGCTCGCAGTGCAGGACAGTTCCGAGAGGCCGCTCAGGGCTGGG
GGGCCTGGTGCACCCCGATGGGGCGGCGGTGCCTGAGCTCGTTTCTTTACTGAGCTC
CCCCCTCCTCCGCCCACTCATCACCCCCTCCCGCGTTGCCACGACAACGCGTAAAAC
TAAAATTCACTTCCCAGTCGCAGGTGGAGACTAGTAGCGCCCCCTCCCCTAGGCCCT
TCCCCGTAGGTCGCGCCCCCATCCCGCCGTCGCTAAGGTGACGGGGAGGGGGCGAC
AGGCGTTGGATCCGGACCGGCCAGGGGTCCTTGGGGGGAAGTCCGGAGCAAACTCC
CCAAACTAGGCTGGCGTGGGGGAGCCGCGCCGAACACGCCAGGGCGCGGTCGAAA
GGGTTAATTCGGAGGGCCTCTCGCCGAGGCGGTGGGAAGGGCCCCGGGCCCTCCCC
GGTCTGGAGGTCCCCGTGGTCCGACCCCCAGGCCTGGGGCGGGGGGGGGTCCCCGC
CATCTCCTCCAGGCCCGAAGCTGGGGGTCGGTGGAGTGGGGGGGAGAAGCCGCCAC
CTCCGGAAATTAATTGTTTTTCTTTCCCCTTCTCGCCCTACCTTGGTCTTCGTGCCCCG
ACGCGGCCCCCACCCCAGCTCCGGGACCCCTTCCTCCGCCGCACCCGCCCCGGTGGT
CCGCGGATGCCCGCCCTTGCCGCTCAGCCACTCCCCCCGCACCGAGGCCTAGGACTC
CCCCCCCCAACCCCGTCACAGCCCCCCAGACCCCCGCCCCGTGGCTCGGCCCCCGCC
CTCCGCACACACCTCCCGCCCCCACCCGGGACCCCGCAAGTAACCCCCCAGCACTGG
CCCTGAGCCCTCCCGGCCCCCGCCTCCGGCGCAGCCCCCTCGCCACCCCCGCTTCCC
TCCCGTCTCAGGCCCCCTCCCCCCGCCGCCCCCGCCCCCGGGGAAGGCAGGCGCCGA
GCTGAGCCGGGGCCGATGCAGCTGAGCCGCGCCGCCGCCGCCGCCGCCGCCGCCCC
TGCGGAGCCCCCGGAGCCGCTGTCCCCCGCGCCGGCCCCGGCCCCGGCCCCCCCCG
GCCCCCTCCCGCGCAGCGCGGCCGACGGGGCTCCGGCGGGGGGGAAGGGGGGGCC
GGGGCGCCGCGCGCGGAGTCCCCGGGCGCTCCGTTCCCCGGCGCGAGCGGCCCCGG
CCCGGGCCCCGGCGCGGGG
Promoter 2-
SEQ ID NO: 2
CATGAGTGTGGGTAATGCAGATTCAGATGTGCACACACACATGCATGAACAC
ACCTCACACGCACATGACGGCAGGTCACACAGACACATGAGCACGGCACTTGCACA
CAGTGGGCTCATATAGAGACACGTGCATGAGCACAGCATCTACACATGACCACGGA
TCTCTCACACACACACGTATGTGCACACCCCCCTCTTTGCTCGCAGCCTTGGGGCTC
ACTTACGCTCTGTGGACGGCTCTGGCCTTGTTTGGACTCCTGGTTCAGCAGCTACTTA
GGAAAGAGATGAGCTGCTGCCCCCTCTTGGGCCCTGACCCACGGAGGTCAGAATTC
CCTACATCATCCCCTCAGCCTTCTTACCTCTGGACCCAGGACTCCAGGATTTGGGGC
CCCCTACTTCCATCTGGACACAACCAGGCCAAGGGGGTGTGTGTGGGCAGAGACTG
GTGACCAGCATGGGTGAGGGCAGTTGAGGCAGGCCAGCTGGAGGCCACGACTGTCC
ATCAGCTCCCGATACTCCCTTCAGAAACGCGGGAGGACCGGACGAAGCGGCTCTTT
CGGCACTACACAGTGGGCTCCTACGACAGCCTCACCTCACACAGGTACGTGCAGGG
ACCCTGGCTGGCGGGAGCATGCATGTGGGACGTGGGGCAGGACCTGCAGTGTAGAG
GGAGGCCAGGTATGAAGAGGTCAGAGGAGGGCAAGGGAATGTGCCTGGCCGACCT
CTCTTCTGCTGCCCGGCCTGTCAGTCCCGGGGTGACTGCAGGGTGGACTGTGGCCGG
CCAACCTCGAGGCAGGGCTTACCTCACTCCTCCCTGCTTTCCTTCATCAGCGATTATG
TCATTGATGACAAAGTGGCTGTCCTGCAGAAACGGGACCACGAGGGCTTTGGTTTTG
TGCTCCGGGGAGCCAAAGGTAATGGGGAGTGGGTGCCCGGGGGTCAGGCAGGCAG
GGGCTGCACACCGCTCAGGGATGTCTTCAGCTTCTTGCTTCTGCCCCCAACTCTGCTC
TGCCCCACACTCCCTCCCTGCCTTGCCCCTGCGGCTCCCGCTTTTGGCCCCGGCTTTC
CTGGTTCCTCTGCTCGGTCGCGACCGTCGTGGTTTGCAACGTCCCGGCCCAGGCGGC
AATTCCTTCCCTTCGTTGTTTTCCACTGTCTTTGCTCTAGAGTCTAACCGAGTGGGGG
CTTTTCTGGCATGGGCAAAGGGAGTTTTGCAGAGGGTCATGTCTAGGATCCTTGGGC
CGGCTCGTCCCTGCGCTGTGGCTCCCCGTGGCCCTCCCCCAGCCCCGGGTCACTGCC
CCCCGCAGTCATCTCTTTCCTGTGCTGAGCCACTCGGAGGTTGCTGTGTGGCAGCCG
CCCCCACCCGAACCTAGCTGGTGAAGCGCCTTCCTAATTGCCCCCCGCAGCAGAGAC
CCCCATCGAGGAGTTCACGCCCACGCCAGCCTTCCCGGCGCTGCAGTATCTCGAGTC
GGTGGACGTGGAGGGTGTGGCCTGGAGGGCCGGGCTGCGCACGGGAGACTTCCTCA
TCGAGGTGAGGTCGTTCTGGCCGGTGCTGCCCAGTGGGATGCTGAGCCCTGAGCCCT
GTGGTGGACGTGCCTGGGGCGTCCCCACCCAGCTGCCTGTCTATCCCAGGTGAACGG
GGTGAACGTGGTGAAGGTCGGACACAAGCAGGTGGTGGCTCTGATTCGCCAGGGTG
GCAACCGCCTCGTCATGAAGGTTGTGTCTGTGACAAGGAAGCCAGAAGAGGACGGG
GCTCGGCGCAGAGGTGAGGGGTCACGCTTCAGGCCTCTGTGCCCAAACTTTCCCCTG
ACCTTAGACCTTTGACTTCAGGCCCACATTCCTCTTCCCATCCCTGACCCCCAACTGC
TGACATCAGATTCCTGAACCTCAATGAACCACCCACACGTGTGGGCTGGGACCCCTC
CCGATCCCTGGTGCTAGACTGCCAGTCCTGTCCCCTGGCCTGAGGCCTTTGACCCCT
GACCCCTGACCTCAGGCTGCCCCTCCTCAGATCCCTGCCCCCTCCTTCCCGGCGTTCC
CCCAGCCCCTGGCGTGGCAGAGCCCTCCTTCCCTACACCGCCAGCAGCTGCCTGGAG
GCCGGGGTTGCCATAGCAACTGTGAGGTTGACGCTGCCG
Promoter 3-
SEQ ID NO: 3
CATGTGTGGATACCCCAGTAGAGGTGTTGGACTCATCAGAGGCTGTCTGGGT
CTCACATGTAGCATTCCTGGGCACACTGGCCATAGCAAAGGGTGTGAGTGGATAAT
GTTGCCCAGGAGAAACTGCAAACTAAGAGGGGAGCTCTCCGAAAATCCATCTAGGA
AGGAAGAGGCCTGGGAAAGAATGTCTCGAAGGGAAACATGAACAGGTCAGCAGCG
GGGTCTTGCTGACCGCCTGGCCAACTGTGGGGCAGAGGGCGGAGTGGGGAGGGTGT
GGCAAGGCCAGACCTCTCTCACTGGCTGAGAGTCACTGGGAAACAGTGAGACGGTG
CAGGATTCAAATGTAGGTGGAAAGACGTGGGGCCGTGGGGGCCTCAGTGTGATCTG
GACTCAGCCTCTTCAGCGTGGCTGCTGGAGGTGTTCGTGGGTGACGGTGCCTGGTGA
AGTATCATGTGTTAGAGGGTGGGTGGCCCAGAAGCTCTCTGGGAGGCTGGCAGGTG
CTGTGATGTGGCTGTTTGGCGGCCGGAGCCTGGCCAGGGAGGTGGCCAGACGGCTG
AGAGTGACAGTGGGTGTGCAGAGAGGCCATGGAAACATGGAAGTCAGACATGGAC
ACTGCTCGGGTTTGTGCCCTGGGGATGGTGTGGGCAGTACCCTCTTCCCCAAGAGAA
AGAGGAGAAGCAGACACCTGGGCCATGGGTGACCCCAGGCCCCTCCCTCCTGTGCC
CTGGGGCCTTTGGCAGGCACAGCCTTCCTTGGGCGTTTTCTCTTGGCCTCCTGACATC
TCCAGCATGCTCTGGGAGGCGGGAGCAATGTGAATGGCTCTCCTCACATCTCTTGCC
CTCGGGCCCCTGGCTCCGTGGGTGCCTCTGGAGCCAGCCTGCCCTCTGCCCAGGTCT
CCAGCTCAGCCCCTGCCAGTACTACCCAGACAGGCACAACTCTGCTCTGCCCAGCTC
GGACCTCCTGGAGCCCAGACCCTGCTCTGGGCTCCGGCCGAGCCTCCTGGCTGCGCT
GCCCGCGCCTGCCCTGTGCCTCTGCCTTCACTGCTGTGGTCCTGCAACTCATCAGTGT
GCTTCAGTCACACCTGAGACTCCAGGTACACCTGCCATCACAACTCCACTGAAAGCT
AAGCCTTGGGGCAGGGGCAGGGCCAGGGTAGGTGTCCATCCTGTGATACAGGTCCC
CAGGCCAGGGCCACTTTGTTGGGGCTGGTGGGTGGAGGCCAGGTGCATATTCCTGGT
CCTGCCCCTCTCTGGGTGTCCACAGTCAGGTCCCTCTGAGTGATTCTCTCGGAGTCTC
CTCTGGCAAGCCCCTCCAGCAGTCACCACCACCTGGGCACCCACCCCCAGGGGACC
CCCCGACCCAACACCACCTGGCACCTCCCCTTGGGAATCCCCCCAACCTTGGCTGCC
TCCCCACTTTGGCCCACTGTGCAGGTGGATCCATGGAGGCCAGGTCCAATGGGAGC
AAAGACAGCTTATTCTGGTGGTTGGTGCCTGTGCTGAGGCCCACCTCAGGTGTTCTT
GAAGCCCCTTTCTGTTTGGGGGCACAGTGTGGCCTCTTCAGGAAGTTCTGCTCTGGA
TCTGAGCAGCTGGGAGGAGGCAGTAGGGACAGAGGCTGAGGAGAGGACAGAAATG
AGGTCCCATGGGAGAGACAGAGCTCAGATCTCCACAGAGGGCTGACCTCTCACAGG
TGGGAGACAAAAGACGAAACCTGCTTTAAGGGGAGCCCCATGCGTGTGTGGGGCAG
GCACTGGTGATGCTGTGTGGTGAGGACAGCCTTTAAGGGGAGCCCCATGCCTGTATG
GGGCAGGGACTGGTGATGCTGTGTGGTGAGGACAGCACAGAGGTTGGGGTGGGGGG
ACTCAGCCTGGGCAGGGGCAGGGAGAGGGGGTGGGAGGGCCACTGGAGAGGGGCC
CGGCCAAAGAGGATGTCAGGGGCTCAGCACCCCCAGGCGGCCAGGCACCTGCCGAG
CACTGCAGCCACGCTTGTGGTTCACCTGCACTTGGAGACTCTCGCACAGGCGCCTCT
CCTCCACCAGCAGCTCCAGCTTTGTCCCTCCCACAGCAGCTGGGCCCTCTGCACCTG
AGTTCCTCCCTCCACA

According to some embodiments, the saRNA may be derived from the promoter of the Mus musculus Shank3 promoter (designated promoter 4 and 5 and set forth in Seq ID NO: 47 and 48.

Promoter 4-
SEQ ID NO: 47
GCTGAGTAATAGGAGGGAGAAGGATGCTTGCAGGAATGATGGATGGAAGGA
AGGAAGGGATACCCACGGATGTGGGAGAGAGGCAAATTACCTAGCAGACAGAGGA
ACCGACAGGGCCAAGCAAGGTCAACCAGAGGGCCAGTGGGTACGAATGGAAGTAT
GAATCACAGGCCAGAGTAAACTTAGGGTGTGGATGTGCTGAGCATGGAGATTTGGG
CATCTCCAGGGGATGCTTAAGTAGTGGACCCATGGAAGTGGCCTGGGGGCAGCTGC
AGGAACAGGGGGCTGAGGACGACTGCTGTGTATTTGCTCCCAATCCCATCCACATGC
CCTGCACTCGTTACAAATCTTTCTCCACCCCCAAGTCTTTTGAAGCTGTGTAACCGAA
AGTGCACCAGGAGGAGGGGAAGAAGTGTGTGGCTAGCCAAAATAAGATCTTTCCAG
AATTTTGGCCCTGGTGAAAGGAAGGAATAAGCAAACCACCCTGGCATTTGAAGGTA
GGGCCAAGGCAGTGTCCTGTCACCTTCCCTGGGAGATAGGGGGTCAGATGGGCCTG
AGGGAGGGCAGCTGGGGCTGCTCAAGTGGCTTGGGAGAGGTAAGGAGGAACCTGA
AGTTATCCTCAACAAAATAATTGCTAAAACACCTCTATCCTATTAATTAGCTCTCCA
GGAACCCATTGGGAAAGACACCCATGTGTTTCTGGGCATTGTTACTGTTGTTGTTTA
GCAGGGAGCAGGCTGGGGCACTGTGAGATTCACTAAGAGTGGGCCAGGCACTGTGC
AGGCCACAGGCTTGATGTCACCCTGGGAGTGTGAAGGCTAGTTGGGGCCAGAGATG
GGGGTCAGTCTGCAGGTAGCTGGTTAAAAAAAATACTTTCTCCCCCCAAAGCTTGGC
AACAAAGATCAAACATAATTAGATTCACTCAGCAAGGGAGGTCAGGGGAGGCTTAA
GGGCTCCCACGAGGTCCAGTTTGGGATAGTAAGCCTGAAAGGGTAAGACCCAGGGC
TCTGGGTCCATTCAGGTCAAGGACATGTCTACAGAGTGCCCAAGGCTGGCCAGCTGC
CTGATTGAGGACATGGGATTCCTCCACTCCTGTCTTATCACTGGTGATCTCTAGCCAT
CATTTTCCACCAGCAGGCAGCTAGTTTAGGGGAAAGGGGAGCTGGAAATAAAAGAT
GGGTGGAGATTTTGATGGGAGGGACTGGGTTTAGCCAGAGGGAGTTGGGGCAGGGG
TCAAGGCGGAGAGGAAAGAGGCAAGGGAGGAAGATGTTAGAGACTCCAGGGTCAG
GAAGCAGAGTTTCCTCTCTGGGTTGGAACACAGGAAACCCAGCCCCGAGCTTCCCA
AGGTGCTTCCTGGAGATAAGGGAGACTCTTTCCTCTCCTGCTCCTCTGACATCCTGCC
CTAAGCCCTAGGGTACCCTAAGTTGCACCACTCAGGCACACCTTGGGAGGGGCTTG
GAGGTCAGAGCCTACTGGGGCTTCACTTCCCCAAGCACGGAGGGGTCCACCCAGGC
TTGAGGGTAGCAGGGAACAGACGTACAGAGGCACTGGAGACTCAGCTCTGCCGGGT
AAGCGGGTTTTTTAAGGGAGGTTCTTTAGGGAGAAGGCTCTCCCTCAAGGGGTAGG
GATGTGGGAGTGAGCCAGGTGGTCCATGGAGGACAATGCTGTGTGGTTGCGATTGG
TCAGAGAGCCCTATATGGAGTCATGAAACATTTTGGGAAAGTGGAGGATCCGTGGG
GGAGTGCAGGCTGTTCTGGGGGGGGGCAGTCTAAGCCAATCACTGTTCTGTGTGGG
GTGAGGGCATTTCTGAGGTGACCCAGGTGGTCAGGGGGTGGGATCTGGGGAGGCTC
TATCGTGTATAGGACCAGGGTAGTCCAGGGTTATTCCTGGGATGCGCAGGAATAGCC
AGTTTTGTGGGCGATCCAGGGTTGCTCACGGAGAAGGTAGGAGAGGCTGTGGCTAA
GGGGATGCTTTCTGCCACTGGCAGAGTTCCCGGCCAGGCTGCTCCACAAAAGCTGTC
CAGTTTGTGCCGCCACCGATGAGTGGCTCTCTGGGGCCTTTAGGGGGTGAGGTGTAA
GTCTAGGTATGCGGGAGAGTGCAGTGGAAGGCAGTAGACACTACCGAGTAGGGCAA
TTCATCCATTCAGGGGCTACACCTTTCTGGGCCTTGGGAGCTAGGCCCCCGTGCGTT
GGGCACTTCCGAGGCAGCGCCGGGCTAAGGCTGGCACGCCACGCGGGCGGCGCCTG
AGTTGGTTCACTCGCAGCGCTCCCCTCCCCGCCCACTCGTCACCCCCTCCCGCGTTGC
CACGACAACGTGTGAAACTAAAATTCAGTTCCCCGCCGCAGGTGGAGACTAGTGGC
GCCCCCTCCCCTAAGCCTTTCCTCGTAGGTCGCGCCCCCAGCCTGCTGTCGCTAAGG
TGACGGGGAGGGGGCGACCAGCTGCAAGGTTGGGCTACCCAAGGTGCGGGAGGCC
CGCAGAGTCCGGACAGACTCCCCAAACTCGGTTGTGGTGGGGGCCGAGAGGAACAC
CCTGGGCGCGGTTAGAAGGGTTGGTCTGGGGGGAGCTCCCGTGGACTGCAGCTCCG
CCCTGCTCGCGCCTGGGGTTTGCAGCCCTCCGACTCCCACCCAGCACCGTGCTCGGG
TGACCCCACCACCTCCTTGGGGTCTGGAGTGGGGTCGGCAGACTGGGGAGATTTCCA
CCCCGGGTCACCAATTTTCCTGCTTGTCTCTCCCTCTCAGTCGGCCTGTGGGTCCTCT
TGCCCCGACGCGTTCCCCACCCAGCTCTAGAACCCTCCGTCACCCCAGTACGGTGGT
CTGCGACGCTCGCCTGCAGCGGAAGCGCGGCGTGCCATCCCCCCGCTGTGTCAGCCA
CTCCCCCTCGACAGCCAACCCCCCCCCTCTTCGGCGCGGGCCCCCGGCCCCCGCCCT
CTGCACCACACGCCCACGCCGCCCCGACCCCGCGGGGGACCCCAGTTTCCCGGCCCC
GGGCTTCCCATCCCCG
Promoter 5-
SEQ ID NO: 48
CTTGTGGTTAGGATGCTCACTACGGCATGAAAACTGGTGGCAAATTGGTTGG
AAATATCTGTGTAGGCTCAAAGCCCATAGGTGAGAGTTATGGCTGTCCGTCGTCCCC
CACATATCTGCCCGAGCCCTGTAGAGTAACAGTACTTTAAACCTCTCTCCTGAGTTCT
GGAATCTTGAGAAACCAGGATATTTGCAGTATCGACCTCCTTATAATCTAAGAGTTG
GAAGCAAAGAACGGAGCCTCTTTCACTCTGTAGGCTTAAGGGGTGGTCACTACGCTG
GTTTCTCTACCAGTACAGTGTAGGGTGTTTCCTTTGAGAAGCCCCCTAGGGTGCCTGT
GAGGGGGTCTTGGACCCTCCTCCAGCACTGCCTATGTTAACCACTGGTTGTACCAAA
TTCCCAGAGTCTTGAGGTTCGTAGCTCTCATTTTTTGCTTCCTGCCGTTTCTCCAGAG
CCAGGTATAAAGAAGGTGTTTGGAAGCCTGTTGGATACAGTGTTACATAAGCAGCTT
GTTGTTTTCACAGTGTCGGCCAGCTCTGATAGCCAGCCCTACGGGTTGATGTTCCCA
CTGGGCTGAGCAGCAAATGCATGCCACTTGGTTAACTGCTGTCGTTTAGTCTGGGCT
AAACGACACTATTATACAGGCTGGTCCCCCAGCCCCATGCCCAGGTTGGGTCAGAG
AGAGTAGACATCGGCTTGTGTGGGTCCAGGGGTTTGGTAGAAGGCCTTTGATGTAGC
ATACACTAGGCTGTTCTTTCTGCAGCATCCCCTTGCCCGTGCAGCCTGATTTCCAGGC
TGTAGACTTACTGGACCCCCAGGGTTGTCTGTCTGCTTGCAAAATCACATTTCTCCTA
TGCAACTTTTGCACGGCCCTTTGGGTTTTCTAAGAATTGAACGGGGTGGTGGCTGGA
AGAATGCATTCAGGGATGTTGCGGTTTGCCAGGAGGACTGTGTGTGAGTGGAACTGT
GTGGCTGTCACTGGGTCCCTTTCAGTTGTGTCTGTTGCAGTTGGCACAGAATAATCTC
CTCTAGTCTAGGCTTTCCAGTTCACCTTTGGGAGGGCAGAATTATTTCTTTTCCACAT
TTATTAGGCGACTGGAATGCCAGGTGTGTGTGTGTGTGCGCGTTCGCGTGTGCGTGC
ACGGCACACGTGTGAGTTCAAGGCTCACCTCTGGCTCCCCACCTCTACCTACTCCCC
ATCACACAGCAGGGGTCCTGACTGGTTCTGAGGAGGTAGCCACCTGGGCTCCTGGCT
GAGAAACACTTAGATGAGTTGTGATACAGGTTAAGGGGGCTTACCTGGTGTGACAG
TGGTCTGCCAGCCAGGATGTGGCAGAGTGTGTTGGGGGTGCTGAGAAGGAAGAGAC
CCTCAGGCAGAGGTTGCCTGTTAAAATAAAAACGAAAAAACAAAAACAAAAACAA
AAAATCCAGGATGTATTCATGGACCCTGGTGGGGCTGCAGTGGCTTCTATTGCTTCC
TGTTGGCTGAGCCTGGCATAGAGCCCAGGACTATGGTCTGCAGGTTCTGGCCCCCAG
CACCAGAGTGAAACCTACAAGGGGCAAATTTGAAGCCGAGGCAGTAACTTAATCTC
TGGCCAAAAGGGCAGTGTCACAGTTGTTTAGGTGGTGGGAACAGTGTAGCATATCTC
TAAGTCTCAAAGAGCCTGGTCATTGGCAGAGCAGGTGAATTTGGCCTGTCGGGGTAC
CAGGGAGCCCAAGCAGGCTTTTGGGTAGAAAGGAAGACCAAGACCTCAGACTCTGT
ATATACCTAAGCCCGAGAAGTGGTCACCATAGATTGACATGGTGGTAGGGACAGCT
GTCCAAGACCACAGGAGTCAGGCCCAGAGGCAGGAGAATTGGGGCTCCCAGTGCAT
GTTGCTGGTCAGGGTGTGAGGAGGCCAGACCACTGCGCCCCGCCTGCCCTTCCCTGG
TGCCCAAAGCCTGGGTGCCAGCCCTTCTCCAGCTCCTACCTGACAGTTCTGCCCGCC
GTTCCCCAGTGCGCCCACCTCTGGGCCCTCGGTGTTTCTTTCCAGCCGCCCGCGTGG
GGTCTGCGGGGGGCACCGCGGACTCAGGGCGCGCCGGCCGCCGCCGCCCGCAGAGG
GAGGAGCTGGCGGAGGAGGCGGGGCGCGGAGCGGCCGTGGCATGGAGC

The term “transcription start site” as used herein refers to a nucleotide marking the transcription start on the template strand of a gene. The transcription start site can appear on the template strand of the promoter region. A gene can have more than one transcription start site.

The term “sequence identity” or “sequence homology” as used herein means that one oligonucleotide strand (sense or antisense) of a saRNA has at least 75% similarity with a region on the coding strand or template strand of the promoter sequence of a target gene.

The term “overhang” as used herein refers to non-base-paired nucleotides at the terminus (5′ or 3′) of an oligonucleotide strand, which is formed by one strand extending out of the other strand in a duplex oligonucleotide. A single-stranded region extending out of the 3′ terminus and/or 5′ terminus of a duplex is referred to as an overhang.

As used herein, the terms “gene activation” or “activating gene expression” can be used interchangeably, and means an increase or upregulation in transcription, translation, expression or activity of a certain nucleic acid as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “small activating RNA,” “saRNA,” and “small activating nucleic acid molecule” can be used interchangeably, and refer to a ribonucleic acid molecule that can upregulate target gene expression. The saRNA can be composed of a first ribonucleic acid strand (antisense strand, also referred to as antisense oligonucleotide strand) containing a ribonucleotide sequence having sequence homology with the non-coding nucleic acid sequence (e.g., a promotor and an enhancer) of a target gene and a second ribonucleic acid strand (sense strand, also referred to as sense oligonucleotide strand) containing a nucleotide sequence complementary with the first ribonucleic acid strand, wherein the first ribonucleic acid strand and the second ribonucleic acid strand form a duplex. The saRNA can also be comprised of a synthesized or vector-expressed single-stranded RNA molecule that can form a hairpin structure by two complementary regions within the molecule, wherein the first region contains a nucleic acid sequence having sequence homology with the target sequence of a promoter of a gene, and a nucleic acid sequence contained in the second region is complementary with the first region. The length of the duplex region of the saRNA molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, or about 50 base pairs. In addition, the terms “saRNA”, “small activating RNA”, and “small activating nucleic acid molecule” also contain nucleic acids other than the ribonucleotide, including, but not limited to, modified nucleotides or analogues.

As used herein, the term “SHANK protein” refers to SH3 and multiple ankyrin repeat domains proteins (SHANKs) SH3 and multiple ankyrin repeat domains 3 (Shank3), also known as proline-rich synapse-associated protein 2 (ProSAP2), is a protein that in humans is encoded by the SHANK3 gene on chromosome 22. The three different SHANK genes can produce multiple protein isoforms that are differentially expressed according to developmental stages, cell types and brain regions. It contains 5 interaction domains or motifs including the ankyrin repeats domain (ANK), a src 3 domain (SH3), a proline-rich domain, a PDZ domain and a sterile a motif domain (SAM). Shank proteins are multidomain scaffold proteins of the postsynaptic density that connect neurotransmitter receptors, ion channels, and other membrane proteins to the actin cytoskeleton and G-protein-coupled signaling pathways. Shank proteins also play a role in synapse formation and dendritic spine maturation. Mutations in this gene are associated with autism spectrum disorder. This gene is often missing in patients with 22q13.3 deletion syndrome (also known as Phelan-McDermid syndrome (PMS). The 22q13.3 deletion encompass the SHANK3 gene and results in SHANK3 haploinsufficiency.

As used herein, the term “synthesis” refers to a method for synthesis of an oligonucleotide, including any method allowing RNA synthesis, such as chemical synthesis, in-vitro transcription, and/or vector-based expression. The present invention provides a method for preparing the small activating nucleic acid molecule, which comprises sequence design and sequence synthesis. The synthesis of the sequence of the small activating nucleic acid molecule can adopt a chemical synthesis or can be entrusted to a biotechnology company specialized in nucleic acid synthesis. Generally speaking, the chemical synthesis comprises the following four steps: (1) synthesis of oligomeric ribonucleotides; (2) deprotection; (3) purification and isolation; (4) desalination and annealing. For example, the steps for chemically synthesizing the double-stranded saRNA, may include:

### (1) Synthesis

Synthesis of 1 micromole of RNA may be set in an automatic DNA/RNA synthesizer (e.g., Applied Biosystems EXPEDITE8909), and the coupling time of each cycle may be set as 10 to 15 minutes. With a solid phase-bonded 5′-O-p-dimethoxytriphenylmethyl-thymidine substrate as an initiator, one base may be bonded to the solid phase substrate in the first cycle, and then, in the nth cycle, one base may be bonded to the base bonded in the n−1 cycle. This process can be repeated until the synthesis of the whole nucleic acid sequence is completed.

### (2) Deprotection

The solid phase substrate bonded with the saRNA is placed into a test tube, and 1 ml of a solution of the mixture of ethanol and ammonium hydroxide (volume ratio: 1:3) is added into the test tube. The test tube is then sealed and incubated at 25-70ºC for 2 to 30 hours. The solution containing the solid phase substrate bonded with the saRNA is filtered, and the filtrate collected. The solid phase substrate is rinsed and the filtrate collected. The eluents are combined and collected, and dried under vacuum for 1 to 12 hours. Then, a solution of tetrabutylammonium fluoride in tetrahydrofuran (1 M) is added. After 4 to 12 hours of standing at room temperature, n-butanol is added. Precipitate is collected to obtain a single-stranded crude product of saRNA by high-speed centrifugation.

### (3) Purification and Isolation

The obtained crude product of saRNA is dissolved in an aqueous ammonium acetate solution with a concentration of 1 mol/ml, and the solution separated by a reversed-phase C18 column of high-pressure liquid chromatography to obtain a purified single-stranded product of saRNA.

### (4) Desalination and Annealing

Salts are removed by gel filtration (size exclusion chromatography). A single sense oligomeric ribonucleic acid strand and a single antisense oligomeric ribonucleic acid strand are mixed into a buffer (10 mM Tris, pH=7.5-8.0, 50 mM NaCl) at a molar ratio of 1:1. The solution is heated to 95ºC, and then slowly cooled to room temperature to obtain a solution containing saRNA.

According to some embodiments, the saRNA is suitable for delivery as naked RNA. Alternatively the saRNA may be delivered using a transport vehicle such as but not limited to a liposome, a conjugated peptide, a delivery molecule, an exosome, a nanoparticle (for example, a polymeric or lipid-based nanoparticle) or the like.

According to some embodiments, the saRNAs may be loaded into exosomes for example utilizing 5′ or 3′ modifications that include hydrophobic molecules, such as cholesterol on any one of the strands or both. According to some embodiments, the saRNAs may be encapsulated by liposomes or other lipid nanoparticles. According to some embodiments, the lipid bodies may themselves be modified in various forms so as to allow for their targeted delivery to a desired site of action, e.g. by exposing the lipid bodies to a neuronal specific membranal antibody resulting in localization to the neurons. Additionally or alternatively, various cell penetrating peptides (CPPs) may be attached covalently or otherwise to the saRNAs so as to facilitate their cellular uptake. CPPs are able to transport different types of cargo molecules across plasma membrane, thus acting as molecular delivery vehicles. According to some embodiments, the CCPs may be or include HIV-TAT, Oligo-Arginine, PEP-1 or the like. Each possibility is a separate embodiment. An additional example of a suitable cell penetrating peptides that are not covalently linked to the saRNAs include a group of secondary amphipathic peptides known as CADY. CADY contains a short peptide sequence of 20 amino acids, having the sequence set forth in SEQ ID NO: 49, namely “Ac-GLWRALWRLLRSLWRLLWRA-cysteamide.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof. According to some embodiments, the term “comprising” may be replaced with the term “consisting of” or consisting essentially thereof.

### EXAMPLES

The present invention is further illustrated by the following examples. These examples are provided merely for illustration purposes and shall not be interpreted to limit the scope or content of the present invention in any way.

### Example 1: Screening of Functional saRNAs Targeting the Promoter Region of SHANK3

### Materials and Methods:

Cell culture—macrophage cells of the bone-merrow-isolated KG-1 cell line were grown in Iscove's Modified Dulbecco's Medium (IMDM) complete growth medium supplemented with 20% (v/v) fetal bovine serum (FBS), under standard incubation conditions.

Transfection—double-stranded RNAs were electroporated at a final concentration of 1 uM or 2 uM to 1,000,000 cell in a total volume of 100 ul using the 4D nucleofector X kit and the FF-100 program of the Nucleofector 4D electroporation system by Lonza. Cells were incubated for 72 h before harvesting. 1 uM Silencer Select siRNA s39916 from Thermo Fisher Scientific was used as a positive control for downregulation of Shank3 expression whilst 0.5 mM Lithium carbonate was used as a positive control for Shank3 upregulation based on the publication by Darville H et. al. 2016

qRT-PCR—RNA was extracted using DirectZol RNA minipreps and treated with DNasel before cDNA synthesis.cDNA was synthesized using the High Capacity cDNA RT kit by Thermo. Shank3 and GAPDH mRNA levels were determined using the following validated Taq-man probes by Thermo: SHANK3: Hs01393541_m1; GAPDH: HS99999905_m1.

FACS—live/dead staining of KG-1 cells was performed 48 hours post transfection, 100 μl samples of post electroporated cells were taken for FACS analysis. Cells were washed with PBS and stained for 10 minutes in the dark with Zombie stain at a final dilution of 1:500 in PBS. Samples were then filter using Nylon mesh and analyzed in FACS. Events were gated for FSC-A vs SSC-A and PB450 intensity.

ELISA—was performed using SHANK3 ELISA Kit (Human) (OKCA00813). Cells were transfected according to the same protocol described herein and with the same saRNAs. Following 72 h-96 h of saRNA treatment cells were collected and protein was extracted according to the kit manufacturer instructions. Samples were run against standards Shank3 samples provided and quantified according to the instruction of the manufacturer.

Computational saRNAs design—The promoter sequences SEQ ID NO:1-3, 47, 48 of SHANK3 were retrieved from the UCSC Genome database to screen for functional saRNAs capable of activating SHANK3 gene expression. Target sequences were obtained by selecting a target with a size of 19 bp starting from the −3 kb position upstream of TSS and moving toward the TSS one base pair (bp) at a time. The target sequences were filtered to remove those that have a GC content higher than 65% or lower than 35% and those that contain 5 or more consecutive nucleotides. After filtration of the target sequences, several dozens of target sequences were found as candidates for further analysis, and these are the sequences listed in Tables 1-5. However, certain bases of the saRNA may optionally be substituted. Optionally the saRNA may further include 5-10 additional 3′ and/or 5′ base pairs. In some embodiments, the saRNA may further include 2-5 additional 3′ and/or 5′ base pairs. While enhancing the complementarity, the stringency of these bases may be lesser than that of the 18-21 nucleotide core.

Double-stranded RNA synthesis—based on these candidate sequences several saRNAs were chemically synthesized at metabion GmbH. The sequences and corresponding SEQ ID NOs of chemically synthesized saRNAs are set forth in Table 6 below. Each saRNA is synthesized with TT 3′ overhang.

### Results

Each of the sense strand and antisense strand double-stranded RNA molecule listed in Table 6 were utilized in the experiment and were 18-21 nucleotides in length, wherein the 18 or 19 nucleotides sequence in the 5′ region of the passenger strand of the double-stranded RNA molecule (e.g., double-stranded saRNA) is 100% homologous with the target sequence of the promoter. The 18 or 19 nucleotides sequence in the 5′ region of the guide strand is fully complementary with the 18 or 19 nucleotides sequence in the 3′ region sequence of the passenger strand (and the promoter target sequence).

TABLE 6
saRNA sequences utilized for targeting the SHANK3 Promoter 1

| | | | | Target | 5′ Modi- | | 3′ DNA |
|---|---|---|---|---|---|---|---|
| | Duplex name | type | Seq ID | promoter | fication | Sequence 5′-3′ | Overhang |
| 1 | Shank3_saRNA_ | Sense | Seq ID6 | Promoter 1 | Phosphate | gagcaaacuccccaaacua | TT |
| | P1.1 | Antisense | | Promoter 1 | Phosphate | uaguuuggggaguuugcuc | TT |
| 2 | Shank3_saRNA_ | Sense | Seq ID 7 | Promoter 1 | Phosphate | caccuccggaaauuaauug | TT |
| | P1.2 | Antisense | | Promoter 1 | Phosphate | caauuaauuuccggaggug | TT |
| 3 | Shank3_saRNA_ | Sense | Seq ID 8 | Promoter 1 | Phosphate | gccugagcucguuucuuua | TT |
| | P1.3 | Antisense | | Promoter 1 | Phosphate | uaaagaaacgagcucaggc | TT |
| 4 | Shank3_saRNA_ | Sense | Seq ID 82 | Promoter 1 | Phosphate | cacugagcucguuucuuuacu | TT |
| | P1.3m | Antisense | | Promoter 1 | Phosphate | aguaaagaaacgagcucagug | TT |
| 5 | Shank3_saRNA_ | Sense | Seq ID 10 | Promoter 1 | Phosphate | gugcuuguggguauuguua | TT |
| | P1.5 | Antisense | | Promoter 1 | Phosphate | uaacaauacccacaagcac | TT |
| 6 | Shank3_saRNA_ | Sense | Seq ID 83 | Promoter 1 | Phosphate | cacucuucugugaacguucuc | TT |
| | P1.17m | Antisense | | Promoter 1 | Phosphate | gagaacquucacagaagagug | TT |
| 7 | Shank3_saRNA_ | Sense | Seq ID 81 | Promoter 1 | Phosphate | ucccgcguugccacgacaa | TT |
| | P1.A | Antisense | | Promoter 1 | Phosphate | uugucguggcaacgcggga | TT |

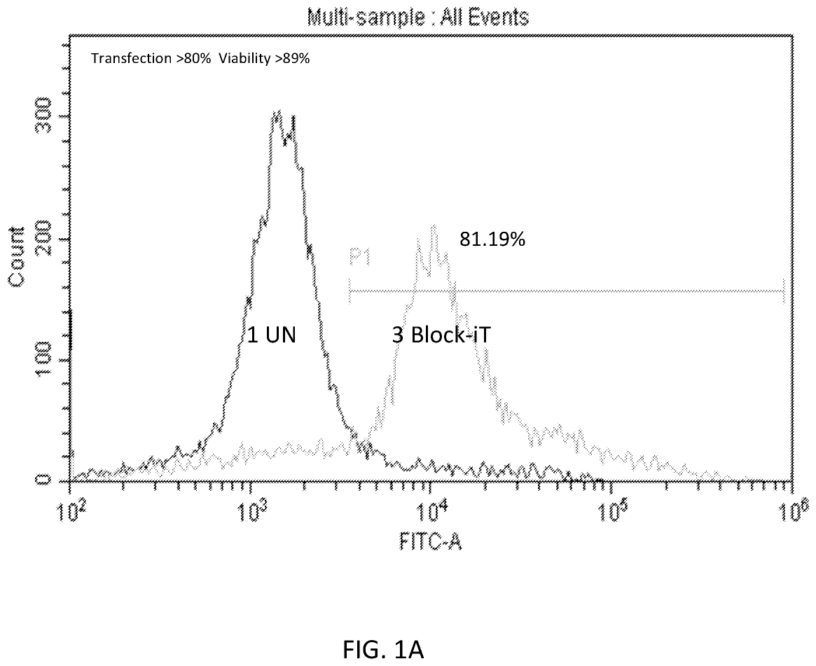

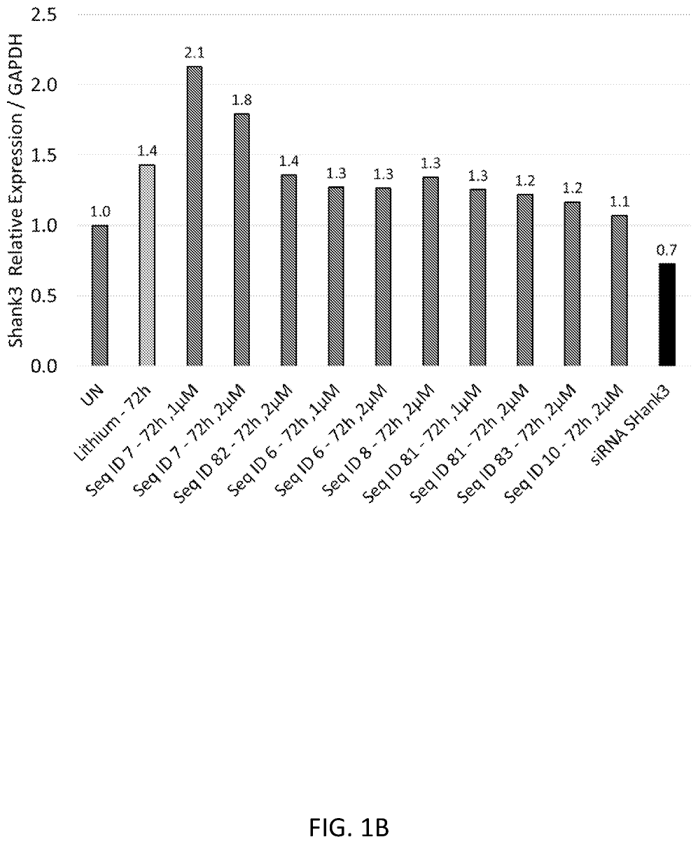

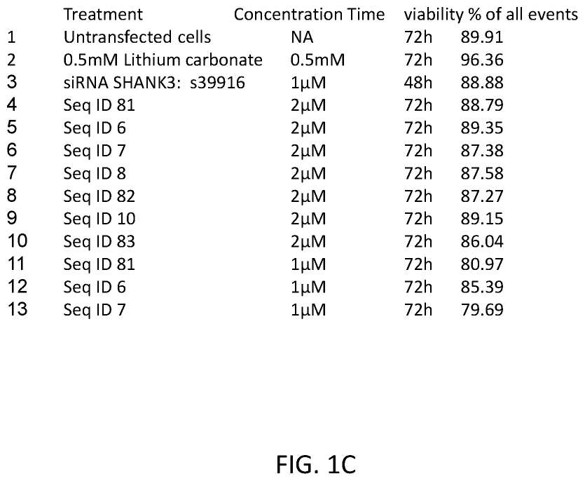

First, the overall transfection efficiency in KG-1 cells was determined using a 4D electroporation system and was estimated to be 81.2% using the Block-IT small fluorescent RNA oligo control (FIG. 1A).

Next, fold change in SHANK3 mRNA level was quantified. Results obtained with positive control siRNA (s39916) reinforce the notion of a successful transfection and suggests that the level of expression of SHANK3 in human KG-1 cells can be regulated using double-stranded RNA, specifically, downregulated to 0.7 fold change relative to untransfected cells.

Results obtained using double-stranded RNA molecules corresponding to SEQ ID NO: 6, 7, 8, 10, 81, 82, and 83 indicate that the saRNA upregulated the activity of promoter 1 in a similar or great magnitude to that achieved by exposing the cells to 0.5 mM lithium bicarbonate, herein serving as a positive control for the induction of SHANK3 mRNA levels (FIG. 1B)

Advantageously, most of the molecules (SEQ ID NO: 6, SEQ ID NO: 8, SEQ ID NO: 10 SEQ ID NO: 81, SEQ ID NO: 82, and SEQ ID NO: 83) increased SHANK3 mRNA levels by 1.2-1.4-fold, relative to untransfected cells. One double-stranded molecule SEQ ID NO: 7 exhibited a much stronger upregulation effect, advantageously, reaching up to 2.1- and 1.8-fold change, relative to untransfected cells, at 1 uM and 2 uM, respectively, which is well above the 1.4-fold change that was expected from treatment with the positive control lithium bicarbonate that exerts a completely different, unspecific, more global effect on gene expression.

These results indicate that saRNA molecules corresponding to SEQ ID NO: 6, 7, 8, 10, 81, 82, and 83 advantageously induce SHANK3 mRNA expression, by complementary base pairing with its promoter 1 corresponding to SEQ ID NO: 1. In particular, SEQ ID NO: 7 increased SHANK3 expression by more than two-fold as compared to untreated control cells and by 1.5 fold as compared to the positive control (lithium).

During the experiment treated KG-1 cells were viable. In a live/dead analysis that was performed by immune-staining of cells followed by FACS sorting, treated cells exhibited similar values to those achieved by un-treated cells (FIG. 1C).

In addition to qrt-PCR, protein level upregulation following saRNA transfection is validated using western-blot and/or sandwich ELISA that allow for EC50 determination for each saRNA molecule. These methods also allow time course analysis of transcriptional upregulation following saRNA transfection for up to 14 days post transfection in terminally differentiated neurons.

### Example 2: saRNA Stability and Immunoreactivity

The stability of saRNAs is evaluated using gel electrophoresis following freeze thaw cycles and/or incubation in human serum.

Immunoreactivity of saRNAs is measured following transfection of saRNAs into human PBMCs and monitoring of TNF-α and IFN-α levels using ELISA.

### Example 3: Shank3 Localization

Shank3 localization to the post synaptic density following transfection with saRNA is evaluated in terminally differentiated neurons using brightfield and fluorescent imaging, and together with neurite growth assays and electrophysiological recordings of network spontaneous calcium oscillations, the efficacy of the saRNA in restoring normal synaptic activity in neurons differentiated for iPSCs of Shank3 haploinsufficency patients can be evaluated.

### Example 4: Toxicity

In addition to the above-mentioned assays, in-vitro toxicity assay using standard proliferation assays such as MTT, enable evaluation of overall toxicity of the Shank3 molecules and total RNA-seq is used to confirm absence of off target up-regulation as a result of saRNA transfection.

### Example 5: In-Vivo Evaluation of Behavioral Effects

In vivo assays for evaluation of the efficacy Shank3 saRNA expression include: biodistribution, ADMA-TOX and multiple behavioral assays including: Ultrasonic vocalizations; Reciprocal dyadic social interaction tests; Repetitive behaviors not during social interaction; Three-chambered social interaction test and the like.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-37. (canceled)

38. A saRNA wherein one strand of the saRNA has at least 75% homology or complementarity with any continuous fragment of 16 to 35 nucleotides in length of a promoter sequence of a human SHANK protein, wherein the saRNA activates or upregulates the expression of the SHANK protein by targeting the human SHANK promoter.

39. The saRNA of claim 38, wherein the saRNA comprises a sense nucleic acid strand and an antisense nucleic acid strand, the sense nucleic acid strand and the antisense nucleic acid strand contain complementary regions capable of forming a double-stranded nucleic acid structure, and the sense nucleic acid strand or the antisense nucleic acid strand has more than 75% homology with any continuous fragment of 16 to 35 nucleotides in length in a sequence of the human SHANK promoter.

40. The saRNA of claim 39, wherein the sense nucleic acid strand and the antisense nucleic acid strand are on the same nucleic acid strand, forming a hairpin single-stranded nucleic acid molecule, wherein the complementary regions of the sense nucleic acid strand and the antisense nucleic acid strand form a double-stranded nucleic acid structure.

41. The saRNA of claim 39, wherein the sense strand has a nucleotide sequence having at least 75% sequence homology to any one of the nucleotide sequences set forth in any of SEQ ID NO: 1-3.

42. The saRNA of claim 39, wherein the antisense strand comprises a nucleotide sequence having at least 90% sequence homology to any one of the nucleotide sequences set forth in SEQ ID NO: 4-46.

43. The saRNA of claim 39, wherein the promotor targeted by the saRNA has a nucleotide sequence with at least 75% sequence homology to the nucleotide sequence set forth in SEQ ID NO: 1 and wherein the antisense strand comprises a nucleotide sequence having at least 90% sequence homology to any one of the nucleotide sequences set forth in SEQ ID NO: 4-24 or SEQ ID NO: 81-83.

44. The saRNA of claim 39, wherein the promotor targeted by the saRNA has a nucleotide sequence with at least 75% sequence homology to the nucleotide sequence set forth in SEQ ID NO: 1 and wherein the antisense strand comprises a nucleotide sequence having at least 90% sequence homology to any one of the nucleotide sequences set forth in SEQ ID NO: 6, 7, 8, 10, 81, 82, and 83.

45. The saRNA of claim 39, wherein the promotor targeted by the saRNA has a nucleotide sequence with at least 75% sequence homology to the nucleotide sequence set forth in SEQ ID NO: 2 and wherein the antisense strand comprises a nucleotide sequence having at least 90% sequence homology to any one of the nucleotide sequences set forth in SEQ ID NO: 25-35.

46. The saRNA of claim 39, wherein the promotor targeted by the saRNA has a nucleotide sequence with at least 75% sequence homology to the nucleotide sequence set forth in SEQ ID NO: 3 and wherein the antisense strand comprises a nucleotide sequence having at least 90% sequence homology to any one of the nucleotide sequences set forth in SEQ ID NO: 36-46.

47. The saRNA of claim 38, wherein the antisense strand further comprises a 3′ having a length of 2-15 nucleotides, and/or wherein the antisense strand comprises a nucleotide analogue.

48. The saRNA of claim 38, further comprising a nuclear localization sequence.

49. A composition comprising one or more of the saRNA molecules of claim 38, and a suitable transport vehicle and/or carrier.

50. The composition of claim 49, wherein the composition is suitable for administration through aerosol.

51. The composition of claim 49, wherein the transport vehicle is a liposome, a conjugated peptide or protein, a delivery molecule, an exosome, nanoparticles, dendrimers, micelles, nanoemulsions and nanosuspensions, a microspheres, and/or cells and/or wherein the carrier is an aqueous solution.

52. The composition of claim 49, wherein the composition is formulated for oral and/or nasal administration, and/or wherein the composition is formulated for administration via inhalation, and/or wherein the composition is formulated for intranasal and/or intrabuccal administration.

53. The composition of claim 49, comprising at least two different saRNA molecules.

54. A method for treating/ameliorating/preventing an intellectual disability, the method comprising administering to a subject in need thereof the saRNA of claim 38, thereby treating/ameliorating/preventing the intellectual disability.

55. The method of claim 54, wherein the intellectual disability is Phelan-McDermid syndrome (PMS) and wherein the method treats and/or ameliorates the symptoms of Phelan-McDermid syndrome, and/or wherein the intellectual disability is idiopathic autism spectrum disorder (ASD) and wherein the method treats and/or ameliorates the symptoms of the ASD, and/or wherein the intellectual disability is schizophrenia and wherein the method treats and/or ameliorates the symptoms of the schizophrenia.

56. The method of claim 54, wherein the subject is a normal subject predisposed to suffer from the intellectual disability.

57. The method of claim 54, further comprising treating a digestive disorder associated with the intellectual disability.


SEQUENCE LISTING (83 sequences)

SEQ ID NO: 1 - DNA, 3000 bp, Homo Sapiens
gatgggagagaagagggggtagacctgggcccagggtggccctgcctggccggtggggtctggtgggccagagggagggtcctcaggaagcttggggaggccgtggggaacctggggattattctcaacaaagtaaggggcaaagacatcctgtgtcccttggcttcctctccaggaacccccccggagcccagaggacgtcatgtgtgtgagtgcttgtgggtattgttatttttgctgtttgccggggtggcggtggagctggggagtgactcaggcttggactcttttcacgatgtgagcatcatgcgtgccaggcactgtgcagaacccaggctcagggcggcactgggcaggggctacggtgttgccccgggcagtgggtctttgacccagctgagtctggggaccatcaatctgtgggctgctggttggtgaagatgtgagattttctctcaaaatgctcagtggccacagccccagtgctggcctggggtggctggattcacctgggctggtgggttggcaggaggcggctggtggggcccagaggtccagtctaagggtcggaggagtgccggccagggtctctgggtccacccagccgagggcaggcttgcagggagacaccaagggagagggaggaggcccagggctgtggaagacctggctgcggagagtccatcactggtggactgggaggccccatccaccaccttctgctggccacagtgggatgaagggccggcacctgggagcaaaggcaggtggaggttgcggtgggagggacggcaaggaaggttagggttagggctgaaggaggggttgggagggagttgggggcctgggtgacagcggggggagagacatgggcaggaagacggtggagacacgaggtgggccgggaagcactggtttcctctctccagggctgggccacaggaaatccagccctcgtgccttccaaggtgcttcctgccgatacgggagcctccagggccccttcctgctccctgctcccgggacccccacccctcccatcctgccccctcccctgggagaccctcagctgcaccactcaggccaggccagtggccttgggaggggcctgtgatgctgggaccacagttcctgggcagggagcaaccgtctaggcgtggggagaacgcaggacgtgacccacacaccgcactggaggctccgctctgcccggtcagtggcgtcctggggaggcgggaggaggaagccagactggtcccagggaagtgaggccctgctctggggttgaccagaggctgttttgaggggagagggctcatcagaaacactctgtcaggggtgctctggggtgagcaggtgctggtcgaggtggatctgctggggagtgggggctgtccttggagggagtgccaagctctgttctgtgtgggtcagggtgtttaggatggtccaggttgtcacgcgggggagggtagggaggtttttctgcgtgtatgggggccggagcagttcaaggttattcctgggaggctcaggcaaaatccgttttgtgggtgatctggagctgctcttggggaggctgggggttagaggctgttttcctccttaggcagaggggttccccaggctgggctcttctgtgaacgttctcaagctctgccctcagggggccagcggctcctctaggaggatctggggctgcttgggggcggaggaggggggtggggaacgccaggcaaagcgggttgggagagttctgcaggaggcggaactatttcgacttaggggctgctcttgggggcacaggggctcgcagtgcaggacagttccgagaggccgctcagggctggggggcctggtgcaccccgatggggcggcggtgcctgagctcgtttctttactgagctcccccctcctccgcccactcatcaccccctcccgcgttgccacgacaacgcgtaaaactaaaattcacttcccagtcgcaggtggagactagtagcgccccctcccctaggcccttccccgtaggtcgcgcccccatcccgccgtcgctaaggtgacggggagggggcgacaggcgttggatccggaccggccaggggtccttggggggaagtccggagcaaactccccaaactaggctggcgtgggggagccgcgccgaacacgccagggcgcggtcgaaagggttaattcggagggcctctcgccgaggcggtgggaagggccccgggccctccccggtctggaggtccccgtggtccgacccccaggcctggggcgggggggggtccccgccatctcctccaggcccgaagctgggggtcggtggagtgggggggagaagccgccacctccggaaattaattgtttttctttccccttctcgccctaccttggtcttcgtgccccgacgcggcccccaccccagctccgggaccccttcctccgccgcacccgccccggtggtccgcggatgcccgcccttgccgctcagccactccccccgcaccgaggcctaggactcccccccccaaccccgtcacagccccccagacccccgccccgtggctcggcccccgccctccgcacacacctcccgcccccacccgggaccccgcaagtaaccccccagcactggccctgagccctcccggcccccgcctccggcgcagccccctcgccacccccgcttccctcccgtctcaggccccctccccccgccgcccccgcccccggggaaggcaggcgccgagctgagccggggccgatgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcgcggagtccccgggcgctccgttccccggcgcgagcggccccggcccgggccccggcgcgggg

SEQ ID NO: 2 - DNA, 2135 bp, Homo sapiens
catgagtgtgggtaatgcagattcagatgtgcacacacacatgcatgaacacacctcacacgcacatgacggcaggtcacacagacacatgagcacggcacttgcacacagtgggctcatatagagacacgtgcatgagcacagcatctacacatgaccacggatctctcacacacacacgtatgtgcacacccccctctttgctcgcagccttggggctcacttacgctctgtggacggctctggccttgtttggactcctggttcagcagctacttaggaaagagatgagctgctgccccctcttgggccctgacccacggaggtcagaattccctacatcatcccctcagccttcttacctctggacccaggactccaggatttggggccccctacttccatctggacacaaccaggccaagggggtgtgtgtgggcagagactggtgaccagcatgggtgagggcagttgaggcaggccagctggaggccacgactgtccatcagctcccgatactcccttcagaaacgcgggaggaccggacgaagcggctctttcggcactacacagtgggctcctacgacagcctcacctcacacaggtacgtgcagggaccctggctggcgggagcatgcatgtgggacgtggggcaggacctgcagtgtagagggaggccaggtatgaagaggtcagaggagggcaagggaatgtgcctggccgacctctcttctgctgcccggcctgtcagtcccggggtgactgcagggtggactgtggccggccaacctcgaggcagggcttacctcactcctccctgctttccttcatcagcgattatgtcattgatgacaaagtggctgtcctgcagaaacgggaccacgagggctttggttttgtgctccggggagccaaaggtaatggggagtgggtgcccgggggtcaggcaggcaggggctgcacaccgctcagggatgtcttcagcttcttgcttctgcccccaactctgctctgccccacactccctccctgccttgcccctgcggctcccgcttttggccccggctttcctggttcctctgctcggtcgcgaccgtcgtggtttgcaacgtcccggcccaggcggcaattccttcccttcgttgttttccactgtctttgctctagagtctaaccgagtgggggcttttctggcatgggcaaagggagttttgcagagggtcatgtctaggatccttgggccggctcgtccctgcgctgtggctccccgtggccctcccccagccccgggtcactgccccccgcagtcatctctttcctgtgctgagccactcggaggttgctgtgtggcagccgcccccacccgaacctagctggtgaagcgccttcctaattgccccccgcagcagagacccccatcgaggagttcacgcccacgccagccttcccggcgctgcagtatctcgagtcggtggacgtggagggtgtggcctggagggccgggctgcgcacgggagacttcctcatcgaggtgaggtcgttctggccggtgctgcccagtgggatgctgagccctgagccctgtggtggacgtgcctggggcgtccccacccagctgcctgtctatcccaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagaggtgaggggtcacgcttcaggcctctgtgcccaaactttcccctgaccttagacctttgacttcaggcccacattcctcttcccatccctgacccccaactgctgacatcagattcctgaacctcaatgaaccacccacacgtgtgggctgggacccctcccgatccctggtgctagactgccagtcctgtcccctggcctgaggcctttgacccctgacccctgacctcaggctgcccctcctcagatccctgccccctccttcccggcgttcccccagcccctggcgtggcagagccctccttccctacaccgccagcagctgcctggaggccggggttgccatagcaactgtgaggttgacgctgccg

SEQ ID NO: 3 - DNA, 2100 bp, Homo sapiens
catgtgtggataccccagtagaggtgttggactcatcagaggctgtctgggtctcacatgtagcattcctgggcacactggccatagcaaagggtgtgagtggataatgttgcccaggagaaactgcaaactaagaggggagctctccgaaaatccatctaggaaggaagaggcctgggaaagaatgtctcgaagggaaacatgaacaggtcagcagcggggtcttgctgaccgcctggccaactgtggggcagagggcggagtggggagggtgtggcaaggccagacctctctcactggctgagagtcactgggaaacagtgagacggtgcaggattcaaatgtaggtggaaagacgtggggccgtgggggcctcagtgtgatctggactcagcctcttcagcgtggctgctggaggtgttcgtgggtgacggtgcctggtgaagtatcatgtgttagagggtgggtggcccagaagctctctgggaggctggcaggtgctgtgatgtggctgtttggcggccggagcctggccagggaggtggccagacggctgagagtgacagtgggtgtgcagagaggccatggaaacatggaagtcagacatggacactgctcgggtttgtgccctggggatggtgtgggcagtaccctcttccccaagagaaagaggagaagcagacacctgggccatgggtgaccccaggcccctccctcctgtgccctggggcctttggcaggcacagccttccttgggcgttttctcttggcctcctgacatctccagcatgctctgggaggcgggagcaatgtgaatggctctcctcacatctcttgccctcgggcccctggctccgtgggtgcctctggagccagcctgccctctgcccaggtctccagctcagcccctgccagtactacccagacaggcacaactctgctctgcccagctcggacctcctggagcccagaccctgctctgggctccggccgagcctcctggctgcgctgcccgcgcctgccctgtgcctctgccttcactgctgtggtcctgcaactcatcagtgtgcttcagtcacacctgagactccaggtacacctgccatcacaactccactgaaagctaagccttggggcaggggcagggccagggtaggtgtccatcctgtgatacaggtccccaggccagggccactttgttggggctggtgggtggaggccaggtgcatattcctggtcctgcccctctctgggtgtccacagtcaggtccctctgagtgattctctcggagtctcctctggcaagcccctccagcagtcaccaccacctgggcacccacccccaggggaccccccgacccaacaccacctggcacctccccttgggaatccccccaaccttggctgcctccccactttggcccactgtgcaggtggatccatggaggccaggtccaatgggagcaaagacagcttattctggtggttggtgcctgtgctgaggcccacctcaggtgttcttgaagcccctttctgtttgggggcacagtgtggcctcttcaggaagttctgctctggatctgagcagctgggaggaggcagtagggacagaggctgaggagaggacagaaatgaggtcccatgggagagacagagctcagatctccacagagggctgacctctcacaggtgggagacaaaagacgaaacctgctttaaggggagccccatgcgtgtgtggggcaggcactggtgatgctgtgtggtgaggacagcctttaaggggagccccatgcctgtatggggcagggactggtgatgctgtgtggtgaggacagcacagaggttggggtggggggactcagcctgggcaggggcagggagagggggtgggagggccactggagaggggcccggccaaagaggatgtcaggggctcagcacccccaggcggccaggcacctgccgagcactgcagccacgcttgtggttcacctgcacttggagactctcgcacaggcgcctctcctccaccagcagctccagctttgtccctcccacagcagctgggccctctgcacctgagttcctccctccaca

SEQ ID NO: 4 - RNA, 19 nt, Artificial Sequence
  saRNA
uuucugaugagcccucucc

SEQ ID NO: 5 - RNA, 19 nt, Artificial
  saRNA
uaaacacccugacccacac

SEQ ID NO: 6 - RNA, 19 nt, Artificial
  saRNA
uaguuuggggaguuugcuc

SEQ ID NO: 7 - RNA, 19 nt, Artificial
  saRNA
caauuaauuuccggaggug

SEQ ID NO: 8 - RNA, 19 nt, Artificial
  saRNA
uaaagaaacgagcucaggc

SEQ ID NO: 9 - RNA, 19 nt, Artificial
  saRNA
gucgaaauaguuccgccuc

SEQ ID NO: 10 - RNA, 19 nt, Artificial
  saRNA
uaacaauacccacaagcac

SEQ ID NO: 11 - RNA, 19 nt, Artificial
  saRNA
uugaugguccccagacuca

SEQ ID NO: 12 - RNA, 19 nt, Artificial
  saRNA
gaguccaagccugagucac

SEQ ID NO: 13 - RNA, 19 nt, Artificial
  saRNA
cacaucuucaccaaccagc

SEQ ID NO: 14 - RNA, 19 nt, Artificial
  saRNA
uaacccuaaccuuccuugc

SEQ ID NO: 15 - RNA, 19 nt, Artificial
  saRNA
aacuguggucccagcauca

SEQ ID NO: 16 - RNA, 19 nt, Artificial
  saRNA
uaaccuugaacugcuccgg

SEQ ID NO: 17 - RNA, 19 nt, Artificial
  saRNA
gaacguucacagaagagcc

SEQ ID NO: 18 - RNA, 19 nt, Artificial
  saRNA
aacggauuuugccugagcc

SEQ ID NO: 19 - RNA, 19 nt, Artificial
  saRNA
gaucacccacaaaacggau

SEQ ID NO: 20 - RNA, 19 nt, Artificial
  saRNA
aaacagccucuaaccccca

SEQ ID NO: 21 - RNA, 19 nt, Artificial
  saRNA
caauuaauuuccggaagug

SEQ ID NO: 22 - RNA, 19 nt, Artificial
  saRNA
uaaagaaacgagcucagug

SEQ ID NO: 23 - RNA, 19 nt, Artificial
  saRNA
aacggauuuugccugagug

SEQ ID NO: 24 - RNA, 19 nt, Artificial
  saRNA
gaacguucacagaagagug

SEQ ID NO: 25 - RNA, 19 nt, Artificial
  saRNA
uugagguucaggaaucuga

SEQ ID NO: 26 - RNA, 19 nt, Artificial
  saRNA
uagagcaaagacaguggaa

SEQ ID NO: 27 - RNA, 19 nt, Artificial
  saRNA
uaugagcccacugugugca

SEQ ID NO: 28 - RNA, 19 nt, Artificial
  saRNA
uagaugcugugcucaugca

SEQ ID NO: 29 - RNA, 19 nt, Artificial
  saRNA
uaaguagcugcugaaccag

SEQ ID NO: 30 - RNA, 19 nt, Artificial
  saRNA
uagggaauucugaccuccg

SEQ ID NO: 31 - RNA, 19 nt, Artificial
  saRNA
gaaucugcauuacccacac

SEQ ID NO: 32 - RNA, 19 nt, Artificial
  saRNA
guguccagauggaaguagg

SEQ ID NO: 33 - RNA, 19 nt, Artificial
  saRNA
gaagcugaagacaucccug

SEQ ID NO: 34 - RNA, 19 nt, Artificial
  saRNA
uuaggaaggcgcuucacca

SEQ ID NO: 35 - RNA, 19 nt, Artificial
  saRNA
caaaggucuaaggucaggg

SEQ ID NO: 36 - RNA, 19 nt, Artificial
  saRNA
uuauccacucacacccuuu

SEQ ID NO: 37 - RNA, 19 nt, Artificial
  saRNA
aagcacacugaugaguugc

SEQ ID NO: 38 - RNA, 19 nt, Artificial
  saRNA
uuucccaggccucuuccuu

SEQ ID NO: 39 - RNA, 19 nt, Artificial
  saRNA
uuugaauccugcaccgucu

SEQ ID NO: 40 - RNA, 19 nt, Artificial
  saRNA
uuccauguuuccauggccu

SEQ ID NO: 41 - RNA, 19 nt, Artificial
  saRNA
uaagcugucuuugcuccca

SEQ ID NO: 42 - RNA, 19 nt, Artificial
  saRNA
uugucucccaccugugaga

SEQ ID NO: 43 - RNA, 19 nt, Artificial
  saRNA
uugugccugucuggguagu

SEQ ID NO: 44 - RNA, 19 nt, Artificial
  saRNA
uucaguggaguugugaugg

SEQ ID NO: 45 - RNA, 19 nt, Artificial
  saRNA
caugauacuucaccaggca

SEQ ID NO: 46 - RNA, 19 nt, Artificial
  saRNA
caagugcaggugaaccaca

SEQ ID NO: 47 - DNA, 3000 bp, Mus musculus
gctgagtaataggagggagaaggatgcttgcaggaatgatggatggaaggaaggaagggatacccacggatgtgggagagaggcaaattacctagcagacagaggaaccgacagggccaagcaaggtcaaccagagggccagtgggtacgaatggaagtatgaatcacaggccagagtaaacttagggtgtggatgtgctgagcatggagatttgggcatctccaggggatgcttaagtagtggacccatggaagtggcctgggggcagctgcaggaacagggggctgaggacgactgctgtgtatttgctcccaatcccatccacatgccctgcactcgttacaaatctttctccacccccaagtcttttgaagctgtgtaaccgaaagtgcaccaggaggaggggaagaagtgtgtggctagccaaaataagatctttccagaattttggccctggtgaaaggaaggaataagcaaaccaccctggcatttgaaggtagggccaaggcagtgtcctgtcaccttccctgggagatagggggtcagatgggcctgagggagggcagctggggctgctcaagtggcttgggagaggtaaggaggaacctgaagttatcctcaacaaaataattgctaaaacacctctatcctattaattagctctccaggaacccattgggaaagacacccatgtgtttctgggcattgttactgttgttgtttagcagggagcaggctggggcactgtgagattcactaagagtgggccaggcactgtgcaggccacaggcttgatgtcaccctgggagtgtgaaggctagttggggccagagatgggggtcagtctgcaggtagctggttaaaaaaaatactttctccccccaaagcttggcaacaaagatcaaacataattagattcactcagcaagggaggtcaggggaggcttaagggctcccacgaggtccagtttgggatagtaagcctgaaagggtaagacccagggctctgggtccattcaggtcaaggacatgtctacagagtgcccaaggctggccagctgcctgattgaggacatgggattcctccactcctgtcttatcactggtgatctctagccatcattttccaccagcaggcagctagtttaggggaaaggggagctggaaataaaagatgggtggagattttgatgggagggactgggtttagccagagggagttggggcaggggtcaaggcggagaggaaagaggcaagggaggaagatgttagagactccagggtcaggaagcagagtttcctctctgggttggaacacaggaaacccagccccgagcttcccaaggtgcttcctggagataagggagactctttcctctcctgctcctctgacatcctgccctaagccctagggtaccctaagttgcaccactcaggcacaccttgggaggggcttggaggtcagagcctactggggcttcacttccccaagcacggaggggtccacccaggcttgagggtagcagggaacagacgtacagaggcactggagactcagctctgccgggtaagcgggttttttaagggaggttctttagggagaaggctctccctcaaggggtagggatgtgggagtgagccaggtggtccatggaggacaatgctgtgtggttgcgattggtcagagagccctatatggagtcatgaaacattttgggaaagtggaggatccgtgggggagtgcaggctgttctgggggggggcagtctaagccaatcactgttctgtgtggggtgagggcatttctgaggtgacccaggtggtcagggggtgggatctggggaggctctatcgtgtataggaccagggtagtccagggttattcctgggatgcgcaggaatagccagttttgtgggcgatccagggttgctcacggagaaggtaggagaggctgtggctaaggggatgctttctgccactggcagagttcccggccaggctgctccacaaaagctgtccagtttgtgccgccaccgatgagtggctctctggggcctttagggggtgaggtgtaagtctaggtatgcgggagagtgcagtggaaggcagtagacactaccgagtagggcaattcatccattcaggggctacacctttctgggccttgggagctaggcccccgtgcgttgggcacttccgaggcagcgccgggctaaggctggcacgccacgcgggcggcgcctgagttggttcactcgcagcgctcccctccccgcccactcgtcaccccctcccgcgttgccacgacaacgtgtgaaactaaaattcagttccccgccgcaggtggagactagtggcgccccctcccctaagcctttcctcgtaggtcgcgcccccagcctgctgtcgctaaggtgacggggagggggcgaccagctgcaaggttgggctacccaaggtgcgggaggcccgcagagtccggacagactccccaaactcggttgtggtgggggccgagaggaacaccctgggcgcggttagaagggttggtctggggggagctcccgtggactgcagctccgccctgctcgcgcctggggtttgcagccctccgactcccacccagcaccgtgctcgggtgaccccaccacctccttggggtctggagtggggtcggcagactggggagatttccaccccgggtcaccaattttcctgcttgtctctccctctcagtcggcctgtgggtcctcttgccccgacgcgttccccacccagctctagaaccctccgtcaccccagtacggtggtctgcgacgctcgcctgcagcggaagcgcggcgtgccatccccccgctgtgtcagccactccccctcgacagccaaccccccccctcttcggcgcgggcccccggcccccgccctctgcaccacacgcccacgccgccccgaccccgcgggggaccccagtttcccggccccgggcttcccatccccg

SEQ ID NO: 48 - DNA, 2149 bp, Mus musculus
cttgtggttaggatgctcactacggcatgaaaactggtggcaaattggttggaaatatctgtgtaggctcaaagcccataggtgagagttatggctgtccgtcgtcccccacatatctgcccgagccctgtagagtaacagtactttaaacctctctcctgagttctggaatcttgagaaaccaggatatttgcagtatcgacctccttataatctaagagttggaagcaaagaacggagcctctttcactctgtaggcttaaggggtggtcactacgctggtttctctaccagtacagtgtagggtgtttcctttgagaagccccctagggtgcctgtgagggggtcttggaccctcctccagcactgcctatgttaaccactggttgtaccaaattcccagagtcttgaggttcgtagctctcattttttgcttcctgccgtttctccagagccaggtataaagaaggtgtttggaagcctgttggatacagtgttacataagcagcttgttgttttcacagtgtcggccagctctgatagccagccctacgggttgatgttcccactgggctgagcagcaaatgcatgccacttggttaactgctgtcgtttagtctgggctaaacgacactattatacaggctggtcccccagccccatgcccaggttgggtcagagagagtagacatcggcttgtgtgggtccaggggtttggtagaaggcctttgatgtagcatacactaggctgttctttctgcagcatccccttgcccgtgcagcctgatttccaggctgtagacttactggacccccagggttgtctgtctgcttgcaaaatcacatttctcctatgcaacttttgcacggccctttgggttttctaagaattgaacggggtggtggctggaagaatgcattcagggatgttgcggtttgccaggaggactgtgtgtgagtggaactgtgtggctgtcactgggtccctttcagttgtgtctgttgcagttggcacagaataatctcctctagtctaggctttccagttcacctttgggagggcagaattatttcttttccacatttattaggcgactggaatgccaggtgtgtgtgtgtgtgcgcgttcgcgtgtgcgtgcacggcacacgtgtgagttcaaggctcacctctggctccccacctctacctactccccatcacacagcaggggtcctgactggttctgaggaggtagccacctgggctcctggctgagaaacacttagatgagttgtgatacaggttaagggggcttacctggtgtgacagtggtctgccagccaggatgtggcagagtgtgttgggggtgctgagaaggaagagaccctcaggcagaggttgcctgttaaaataaaaacgaaaaaacaaaaacaaaaacaaaaaatccaggatgtattcatggaccctggtggggctgcagtggcttctattgcttcctgttggctgagcctggcatagagcccaggactatggtctgcaggttctggcccccagcaccagagtgaaacctacaaggggcaaatttgaagccgaggcagtaacttaatctctggccaaaagggcagtgtcacagttgtttaggtggtgggaacagtgtagcatatctctaagtctcaaagagcctggtcattggcagagcaggtgaatttggcctgtcggggtaccagggagcccaagcaggcttttgggtagaaaggaagaccaagacctcagactctgtatatacctaagcccgagaagtggtcaccatagattgacatggtggtagggacagctgtccaagaccacaggagtcaggcccagaggcaggagaattggggctcccagtgcatgttgctggtcagggtgtgaggaggccagaccactgcgccccgcctgcccttccctggtgcccaaagcctgggtgccagcccttctccagctcctacctgacagttctgcccgccgttccccagtgcgcccacctctgggccctcggtgtttctttccagccgcccgcgtggggtctgcggggggcaccgcggactcagggcgcgccggccgccgccgcccgcagagggaggagctggcggaggaggcggggcgcggagcggccgtggcatggagc

SEQ ID NO: 49 - RNA, 19 nt, Artificial
  saRNA
uuuuaaccagcuaccugca

SEQ ID NO: 50 - RNA, 19 nt, Artificial
  saRNA
uuccuuccuuucaccaggg

SEQ ID NO: 51 - RNA, 19 nt, Artificial
  saRNA
uuacucuggccugugauuc

SEQ ID NO: 52 - RNA, 19 nt, Artificial
  saRNA
uuagugaaucucacagugc

SEQ ID NO: 53 - RNA, 19 nt, Artificial
  saRNA
uauuuccagcuccccuuuc

SEQ ID NO: 54 - RNA, 19 nt, Artificial
  saRNA
uuaggguacccuagggcuu

SEQ ID NO: 55 - RNA, 19 nt, Artificial
  saRNA
uucaugacuccauauaggg

SEQ ID NO: 56 - RNA, 19 nt, Artificial
  saRNA
uucaggcuuacuaucccaa

SEQ ID NO: 57 - RNA, 19 nt, Artificial
  saRNA
uagacauguccuugaccug

SEQ ID NO: 58 - RNA, 19 nt, Artificial
  saRNA
uugcccuacucgguagugu

SEQ ID NO: 59 - RNA, 19 nt, Artificial
  saRNA
gacaagcaggaaaauuggu

SEQ ID NO: 60 - RNA, 19 nt, Artificial
  saRNA
uacccugguccuauacacg

SEQ ID NO: 61 - RNA, 21 nt, Artificial
  saRNA
uauuccuuccuuucaccagug

SEQ ID NO: 62 - RNA, 21 nt, Artificial
  saRNA
guuuacucuggccugugagug

SEQ ID NO: 63 - RNA, 21 nt, Artificial
  saRNA
guuucaugacuccauauagug

SEQ ID NO: 64 - RNA, 21 nt, Artificial
  saRNA
uguagacauguccuugaagug

SEQ ID NO: 65 - RNA, 21 nt, Artificial
  saRNA
gaauugcccuacucgguagug

SEQ ID NO: 66 - RNA, 59 nt, Artificials
  misc_feature (1)..(59): n is a, c, g, or u
nnnnnnnnnnnnnnnnnnnnnnnnnnnnnnnnnnnnnnnnnnnnnnnnnnnnnnnnnnn

SEQ ID NO: 67 - RNA, 19 nt, Artificial
  saRNA
uuuuaacaggcaaccucug

SEQ ID NO: 68 - RNA, 19 nt, Artificial
  saRNA
uuccaaccaauuugccacc

SEQ ID NO: 69 - RNA, 19 nt, Artificial
  saRNA
uacuguuacucuacagggc

SEQ ID NO: 70 - RNA, 19 nt, Artificial
  saRNA
uuagaaaacccaaagggcc

SEQ ID NO: 71 - RNA, 19 nt, Artificial
  saRNA
uauucugugccaacugcaa

SEQ ID NO: 72 - RNA, 19 nt, Artificial
  saRNA
uuugccccuuguagguuuc

SEQ ID NO: 73 - RNA, 19 nt, Artificial
  saRNA
uaaggaggucgauacugca

SEQ ID NO: 74 - RNA, 19 nt, Artificial
  saRNA
uacugguagagaaaccagc

SEQ ID NO: 75 - RNA, 19 nt, Artificial
  saRNA
uauaccuggcucuggagaa

SEQ ID NO: 76 - RNA, 19 nt, Artificial
  saRNA
caaucuauggugaccacuu

SEQ ID NO: 77 - RNA, 19 nt, Artificial
  saRNA
uuuuaacaggcaaccagug

SEQ ID NO: 78 - RNA, 19 nt, Artificial
  saRNA
uauaccuggcucuggagug

SEQ ID NO: 79 - RNA, 19 nt, Artificial
  saRNA
caaucuauggugaccagug

SEQ ID NO: 80 - RNA, 59 nt, Artificial; identical to SEQ ID NO: 66
  saRNA
  misc_feature (1)..(59): n is a, c, g, or u

SEQ ID NO: 81 - RNA, 19 nt, Artificial
  saRNA
uugucguggcaacgcggga

SEQ ID NO: 82 - RNA, 21 nt, Artificial
  saRNA
aguaaagaaacgagcucagug

SEQ ID NO: 83 - RNA, 21 nt, Artificial
  saRNA
gagaacguucacagaagagug